SEQUENCE LISTING (4 sequences)

SEQ ID NO: 1 - protein, 137 aa, Homo sapiens
MGLTSQLLPPLFFLLACAGNFVHGHKCDITLQEIIKTLNSLTEQKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 2 - protein, 140 aa, Mus musculus
MGLNPQLVVILLFFLECTRSHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 3 - protein, 147 aa, Rattus norvegicus
MGLSPHLAVTLFCFLICTGNGIHGCNDSPLREIINTLNQVTEKGTPCTEMFVPDVLTATRNTTENELICRASRVLRKFYFPRDVPPCLKNKSGVLGELRKLCRGVSGLNSLRSCTVNESTLTTLKDFLESLKSILRGKYLQSCTSMS

SEQ ID NO: 4 - protein, 825 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

(19) United States
(12) Patent Application Publication
(10) Pub. No.: US 2024/0336688 A1
(43) Pub. Date: Oct. 10, 2024

(54) DUAL-BLOCKADE OF IL-4 SIGNALING AND IMMUNE CHECKPOINT PROTEINS FOR TREATING CANCER

(71) Applicant: Icahn School of Medicine at Mount Sinai, New York, NY (US)

(72) Inventors: Miriam Merad, New York, NY (US); Thomas U. Marron, New York, NY (US)

(73) Assignee: Icahn School of Medicine at Mount Sinai, New York, NY (US)

(21) Appl. No.: 18/580,852

(22) PCT Filed: Jul. 22, 2022
(86) PCT No.: PCT/US2022/074053

§ 371 Date: Jan. 19, 2024

Related U.S. Application Data
(60) Provisional application No. 63/224,837, filed on Jul. 22, 2021.

(51) Int. Cl. C07K 16/28; A61P 35/00

(52) U.S. Cl. CPC C07K 16/2827; A61P 35/00; C07K 16/2818; C07K 2317/24

(57) ABSTRACT

The present disclosure provides for cancer therapies that comprise administering inhibitors of IL4 signaling to a subject in need thereof, particularly in combination with an immune checkpoint inhibitor. In a particular embodiment, the present inventors have discovered that immunotherapies that block the PD-1/PD-L1 immune checkpoint axis may be made more effective with the co-administration of a specific dosing regimen of an antibody that disrupts IL-4 signaling, wherein the dosing regimen comprises about 600 mg of dupilumab as a first dose, followed by two subsequent doses of about 300 mg of dupilumab about every three weeks thereafter. Other dosing regimens are within the scope of the disclosure and are described herein.

### RELATED-APPLICATIONS

This application claims the benefit of U.S. Provisional Patent Application Ser. No. 63/224,837, filed Jul. 22, 2021, entitled, “Dual-Blockade of IL-4 Signaling and Immune Checkpoint Proteins For Treating Cancer,” which is incorporated herein by reference in its entirety.

### FIELD OF THE INVENTION

The present disclosure relates to the field of cancer therapy. Specifically, the present disclosure relates to methods of treating cancer involving activating anti-tumor immunity by administering inhibitors of IL4 signaling to a subject in need thereof, particularly in combination with an immune checkpoint inhibitor.

### BACKGROUND

An important function of the immune system is its ability to tell between normal cells in the body and those it sees as “foreign.” This lets the immune system attack the foreign cells while leaving the normal cells alone. To do this, it uses “checkpoints.” Immune checkpoints are proteins on certain immune cells (e.g., the surface of T cells) that need to be activated (or inactivated) to start an immune response. Cancer cells sometimes find ways to use these checkpoints to avoid being attacked by the immune system. Drugs that target these checkpoints are called checkpoint inhibitors and are widely used in the treatment of cancer. However, many patients do not respond to such drugs and/or patients may develop resistance to checkpoint immunotherapies.

In more detail, immune checkpoints exist as an important component of the immune system. In general, the physiological role of immune checkpoints is to prevent the development of an immune response from being so strong that it destroys healthy cells in the body. Immune checkpoints engage when proteins on the surface of T cells recognize and bind to partner proteins on other cells, such as some tumor cells. These proteins are called immune checkpoint proteins. Examples of such proteins include cytotoxic T-lymphocyte-associated protein 4 (CTLA-4), programmed cell death protein 1 (PD-1), programmed cell death ligand 1 (PD-L1), and programmed cell death ligand 2 (PD-L2). When the immune checkpoint and partner proteins bind together, they initiate signaling to inhibit the related immune response, thus acting as an “off” signal to the T cells. Due to the inhibitory activity of these proteins, they are also sometimes referred to as blockades or checkpoint blockades. Activation of these checkpoints can prevent and/or inhibit the immune reaction and hinder the immune system from destroying the cancer (e.g., cancer cells, tumors).

Immunotherapy drugs called immune checkpoint inhibitors work by blocking checkpoint proteins from binding with their partner proteins. This prevents the “off” signal from being sent, allowing the T cells to kill cancer cells (e.g., inhibiting or blocking the inhibitory activity of the checkpoint proteins). Thus, immune checkpoint inhibitors—which can also be referred to as checkpoint blockade therapies—are anti-cancer immunotherapies that block the inhibitory checkpoint molecules thereby reactivating the immune response against the tumor. In this way, checkpoint inhibitors treat cancer by indirectly activating the immune response—which is otherwise blocked by the immune checkpoints—to kill cancer cells.

The emergence of immune checkpoint inhibitors, such as blocking antibodies against CTLA-4, PD-1, PD-L1, and PD-L2, have dramatically transformed cancer care over the last decade over a wide range of tumor types. However, despite these promising long-term responses, the majority of patients fail to respond to immune checkpoint blockade, demonstrating primary resistance. Additionally, many of those who initially respond to treatment eventually experience relapse secondary to acquired resistance. Both primary and acquired resistance are a result of complex and constantly evolving interactions between cancer cells and the immune system. Various mechanisms of resistance have been characterized to date, and more continue to be uncovered. By elucidating and targeting mechanisms of resistance, treatments can be tailored to improve clinical outcomes.

Medical solutions for reversing and/or avoiding resistance to immune checkpoint inhibitors in the treatment of cancer would be a significant advance in the art.

### SUMMARY

The present disclosure relates to a novel improvement in the use of immune checkpoint inhibitors (e.g., agents that block the PD-1/PD-L1 immune checkpoint axis) in the treatment of cancer (e.g., non-small cell lung cancer, NSCLC). In many instances, despite the success of immune checkpoint immunotherapies, not all patients respond to such agents and/or the cancer being treated can develop resistance to such immunotherapies. Without being bound by theory, the present inventors have discovered that adjusting the tumor microenvironment with agents which block or otherwise inhibit IL-4 signaling can render a tumor sensitive to an immune checkpoint immunotherapy. In a particular embodiment, the present inventors have discovered that immunotherapies that block the PD-1/PD-L1 immune checkpoint axis may be made more effective with the co-administration of a specific dosing regimen of an antibody that disrupts IL-4 signaling, wherein the dosing regimen comprises about 600 mg of dupilumab as a first dose, followed by two subsequent doses of about 300 mg of dupilumab about every three weeks thereafter. Other dosing regimens are within the scope of the disclosure and are described herein.

Without being bound by theory, inflammation is a hallmark of cancer and chronically inflamed tumor microenvironments have been shown to contribute to the growth of and the evasion of the immune system by solid tumors. The tumor microenvironment is often characterized by a complex inflammatory milieu that favors the recruitment and differentiation of suppressive immune cells such as regulatory T cells and macrophages^{3,4}, and dampens the activity of T effector cells and dendritic cells, which are instrumental in presenting tumor-associated antigen to T cells and thereby initiating tumor-directed adaptive immune responses⁵⁻⁷. Those cells are being skewed towards suppressive phenotypes by anti-inflammatory cytokines present in the tumor microenvironment such as IL-10, TGF-β and Th2 cytokines^{8,9}. IL-4 and IL-13 are the primary mediators of Th2 immune responses and are required to generate immune responses against parasite infections. However, they are also secreted by stressed or dying epithelial cells, and therefore are induced in many solid tumors^{10,11}.

In a mouse model of lung adenocarcinoma (Kras^{G12D}; Tp53−/−), the inventors recently identified an IL-4-driven regulatory module in tumor antigen-charged dendritic cells, indicating IL-4 signaling played an important role in mediating immuno-suppression in this tumor model (FIG. 2A). IL-4 antibody blockade in tumor-bearing mice in vivo led to increased IL-12 production in dendritic cells, a key cytokine produced by mature dendritic cells to activate T cells (FIG. 2B). Subsequently, effector cytokines IFNγ and TNF in CD8+ T cells were increased in mice treated with IL-4 blockade (FIG. 2C) and importantly this led to a significant decrease in tumor burden (FIG. 2D). As a monotherapy, IL-4 antibody blockade also significantly decreased the growth of other murine tumor models, such as B16 melanoma, B16 lung metastasis and KP1 lung adenocarcinoma. In the KP1 lung adenocarcinoma model, combination of PD-L1 blockade and IL-4 antibody blockade decreased tumor load compared to either monotherapy alone. See FIG. 4.

The inventors' preclinical data provides strong evidence that blocking Th2 immune mediators, and specifically IL-4 signaling, may be beneficial for cancer patients by activating dendritic cells and subsequent T effector cells generating a robust immune response against tumor antigens.

In addition, the inventors found that Th2 skewing of the tumor microenvironment might impair the patients' response to immunotherapy. Therefore, the inventors' instant disclosure provides for a method that combines IL-4 blockade with PD-1/PD-L1 blockade for treating cancer, e.g., non-small cell lung cancer (NSCLC). In a particular embodiment, the present inventors have discovered that immunotherapies that block the PD-1/PD-L1 immune checkpoint axis may be made more effective with the co-administration of a specific dosing regimen of an antibody that disrupts IL-4 signaling, wherein the dosing regimen comprises about 600 mg of dupilumab as a first dose, followed by two subsequent doses of about 300 mg of dupilumab about every three weeks thereafter. Other dosing regimens are within the scope of the disclosure and are described herein.

In some aspects, the disclosure relates to a method of treating cancer comprising administering to a subject in need thereof a therapeutically effective amount of (1) an anti-IL4 signaling antibody and (2) an immune checkpoint inhibitor. In some embodiments, the immune checkpoint inhibitor is an anti-CTLA-4 therapy selected from the group consisting of ipilimumab, tremelimumab, and combinations thereof. In some embodiments, the immune checkpoint inhibitor is ipilimumab.

In some embodiments, the immune checkpoint inhibitor is an anti-PD-1 therapy selected from the group consisting of nivolumab, pembrolizumab, pidilizumab, MEDI0680, and combinations thereof. In some embodiments, the immune checkpoint inhibitor is an anti-PD-L1 therapy selected from the group consisting of atezolizumab, BMS-936559, MEDI4736, MSB0010718C, and combinations thereof.

In some embodiments, the anti-IL4 signaling antibody is an anti-IL4 antibody or antigen-binding fragment. In some embodiments, the anti-IL4 antibody specifically binds human IL4 comprising the amino acid sequence of SEQ ID NO: 1. In some embodiments, the anti-IL4 signaling antibody is a human or humanized antibody. In some embodiments, the anti-IL4 signaling antibody is an IgG. In some embodiments, the anti-IL4 signaling antibody is an IgG, IgG1, IgG2, IgG2A, IgG2B, IgG2C, IgG3, IgG4, IgA1, IgA2, IgD, IgM, and IgE constant domains. In some embodiments, the anti-IL4 signaling antibody is an anti-IL4 receptor antibody or antigen-binding fragment. In some embodiments, the anti-IL4 receptor antibody specifically binds human IL4 receptor comprising the amino acid sequence of SEQ ID NO: 1.

In some embodiments, the treatment is administered intravenously.

In some embodiments, the method further comprises administering an effective amount of an immune checkpoint inhibitor.

In some embodiments, the immune checkpoint inhibitor is an anti-CTLA-4 antibody. In some embodiments, the immune checkpoint inhibitor is selected from the group consisting of ipilimumab, tremelimumab, nivolumab, pembrolizumab, pidilizumab, MEDI0680, atezolizumab, BMS-936559, MEDI4736, MSB0010718C. and combinations thereof.

In some aspects, the disclosure relates to a method of synergistically increasing the activity of an immune checkpoint inhibitor. In some embodiments, the immune checkpoint inhibitor is an anti-CTLA-4 therapy selected from the group consisting of ipilimumab, tremelimumab, and combinations thereof. In some embodiments, the immune checkpoint inhibitor is ipilimumab.

In some embodiments, the immune checkpoint inhibitor is an anti-PD-1 therapy selected from the group consisting of nivolumab, pembrolizumab, pidilizumab, MEDI0680, and combinations thereof. In some embodiments, the immune checkpoint inhibitor is an anti-PD-L1 therapy selected from the group consisting of atezolizumab, BMS-936559, MEDI4736, MSB0010718C, and combinations thereof.

In some aspects, the disclosure relates to a pharmaceutical composition comprising: (a) a therapeutically effective amount of an anti-IL-4 signaling antibody and (b) a therapeutically effective amount of at least one immune checkpoint inhibitor; and (c) at least one pharmaceutically-acceptable carrier. In a particular embodiment, the present inventors have discovered that immunotherapies that block the PD-1/PD-L1 immune checkpoint axis may be made more effective with the co-administration of a specific dosing regimen of an antibody that disrupts IL-4 signaling, wherein the dosing regimen comprises about 600 mg of dupilumab as a first dose, followed by two subsequent doses of about 300 mg of dupilumab about every three weeks thereafter. Other dosing regimens are within the scope of the disclosure and are described herein.

In some embodiments, the immune checkpoint inhibitor is selected from the group consisting of ipilimumab, tremelimumab, nivolumab, pembrolizumab, pidilizumab, MEDI0680, atezolizumab, BMS-936559, MEDI4736, MSB0010718C. and combinations thereof. In some embodiments, the immune checkpoint inhibitor is a therapy selected from the group consisting of anti-CTLA-4, anti-PD-1, anti-PD-L1, and combinations thereof.

In some aspects, the disclosure relates to a method of immunotherapy comprising administering to a subject in need thereof a pharmaceutical composition comprising a therapeutically effective amount of an anti-IL-4 signaling antibody.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 provides a schematic showing an embodiment of the disclosure involving dual-blockage of IL-4 signaling (e.g., dupilumab) and checkpoint proteins (e.g., anti-PD-(L)1 antibodies or other immune checkpoint inhibitors) for treating cancer (e.g., relapsed/refractory NSCLC). In the embodiment shown, the treatment comprises standard of care anti-PD-(L)1 antibodies (pembrolizumab (KEYTRUDA®) combined with a first dose of 600 mg of dupilumab (anti-IL4 signaling antibody) at about day 1, followed by two successive doses of 300 mg of dupilumab every three weeks or so.

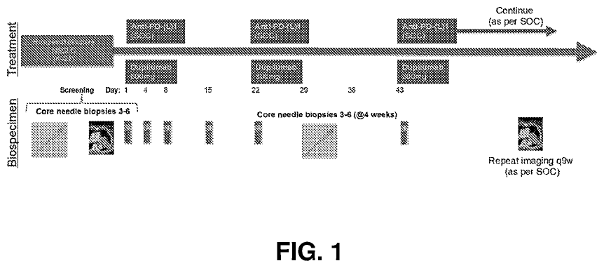

FIG. 2 demonstrates IL-4 blockade increases immune effector functions and reduces tumor growth. Mice were injected with 105 tumor cells and lungs were harvested on day 28. (FIG. 2A), single cell sequencing of dendritic cell subsets (mregDC=mature DC enriched in regulatory molecules) showing average cluster expression of IL-4 response genes. (FIG. 2B and FIG. 2C), lungs were digested for flow cytometry and dendritic cells (FIG. 2A) and T cells (FIG. 2C) were stained. (FIG. 2D), mice were treated with IL-4 blocking antibody (aIL4) on days 22, 24 and 26. FFPE lungs were stained with H&E and tumors were quantified manually. (FIG. 2E), mice were treated with either aIL4, PD-L1 blocking antibody (aPD-L1) or both (combo) on days 22, 24 and 26. Lungs were stained with H&E or digested for flow cytometry.

FIG. 3 provides a schematic showing the targeting by dupilumab of the alpha subunit of the IL-4R of the Type I and Type II receptors, which blocks IL-4 signaling in the case of Type I receptors, and IL-4 and IL-13 signaling in the case of Type II receptors.

FIG. 4 demonstrates that a combination of PD-L1 blockade and IL-4 antibody blockade decreased tumor load compared to either monotherapy alone. 8-week old male C57BL/6 mice were injected intravenously with 150,000 HKP1-mCherry cells on Day 0. Starting on Day 1, mice were treated every other day with 200 μg αPD-L1, 25 μg αIL-4, or a combination of both antibodies intraperitoneally. Lung tumor burden was analyzed by histology on Day 18.

### DETAILED DESCRIPTION

The inventors discovered that adjusting the tumor microenvironment with agents which block or otherwise inhibit IL-4 signaling can render a tumor sensitive to an immune checkpoint immunotherapy. In a particular embodiment, the inventors discovered that immunotherapies that block the PD-1/PD-L1 immune checkpoint axis may be made more effective with the co-administration of a specific dosing regimen of an antibody that disrupts IL-4 signaling, wherein the dosing regimen comprises about 600 mg of dupilumab (marketed as DUPIXENT®) as a first dose, followed by two subsequent doses of about 300 mg of dupilumab about every three weeks thereafter. Other dosing regimens are within the scope of the disclosure and are described herein.

Immunotherapy represents a paradigm shift in the treatment of cancer, particularly the inhibition of various checkpoints that control host T cell activity. Programmed cell death 1 (PD-1) interacting with its corresponding ligand PD-L1 (produced by various cancer cells) has emerged as a common means by which cancer cells evade the host immune response (1). For example, PD-1 is expressed on activated CD8+ T cells (as well as B cells and natural killer cells) in the setting of chronic antigen exposure. The interaction between PD-1 and PD-L1 or PD-L2 on host tissues leads to the inhibition of T cell receptor (TCR) signaling and CD28 co-stimulation (2, 3), limits T cell interactions with target cells, and ultimately leads to their inactivation and loss of proliferative capacity (4-7). PD-L1 expression is induced by localized inflammatory stimuli, such as interferons (IFNs) released by the infiltrating T cells (5). The PD-L1 induction process in cancer has been termed “adaptive immune resistance” (1) and represents a mechanism by which cancer cells protect themselves from T cell-mediated destruction.

This approach to treating cancer has led to the clinical development of therapeutic monoclonal antibodies blocking PD-1 or PD-L1. Pembrolizumab (KEYTRUDA®) and nivolumab (OPDIVO®) are two such agents targeting PD-1, while atezolizumab (TECENTRIQ®), avelumab (BAVENCIO®), and durvalumab (IMFINZI®) block PD-L1 instead (8). Collectively, these agents are approved for the treatment of a wide variety of malignancies, including metastatic melanoma, non-small-cell lung cancer (NSCLC), head and neck squamous cell cancer, Hodgkin's lymphoma, renal cell carcinoma (RCC), urothelial carcinoma, Merkel cell carcinoma, gastric carcinoma, and hepatocellular carcinomas (8-22). These agents inhibit the negative regulatory effects of PD-L1 on patient T cells via PD-1, resulting in the enhancement of a pre-existent antitumor immune activity. This unleashes a focused T cell response against a patient's tumor, as it increases proliferation of tumor infiltration lymphocytes (TILs) and leads to a more clonal TCR repertoire within the T cell population directed against the tumor (23). These effects ultimately provide patients with significant and durable immune responses against their malignancy, with complete responses lasting for years in some cases.

Although PD-1/PD-L1 checkpoint blockade can result in dramatic therapeutic responses, this therapy is only effective in a subset of patients, and many patients are only partial responders to therapy (9, 12, 24). Patients who do not respond to initial therapy with PD-1/PD-L1 blockade are referred to as having “primary resistance” to therapy (25). Furthermore, there is a growing subset of patients who, despite showing a robust initial response to therapy, go on to develop progressive disease. This phenomenon, in which disease is either refractory to resumption of therapy, or develops despite continuation of therapy, is known as “acquired resistance” to PD-1/PD-L1 blockade immunotherapy (25, 26). Both phenomena are highly complex, as the mechanisms for both types of resistance can be overlapping and/or multifactorial. Furthermore, each patient's individual environmental and genetic factors, as well as prior treatments, can create an evolving therapeutic landscape unique to a given patient. As the mechanisms responsible for both primary and acquired resistance to PD-1/PD-L1 blockade are further elucidated, improved treatments with superior therapeutic efficacy can be developed. This disclosure provides an approach to overcome such resistance.

The inventors' preclinical data provides strong evidence that blocking Th2 immune mediators, and specifically IL-4 signaling, may be beneficial for cancer patients by activating dendritic cells and subsequent T effector cells generating a robust immune response against tumor antigens. In addition, the inventors hypothesize that Th2 skewing of the tumor microenvironment might impair the patients' response to immunotherapy. Therefore, the disclosure provides a method of combining IL-4 blockade with PD-1/PD-L1 blockade as an effective anti-cancer therapy. In a particular embodiment, the inventors discovered that immunotherapies that block the PD-1/PD-L1 immune checkpoint axis may be made more effective with the co-administration of a specific dosing regimen of an antibody that disrupts IL-4 signaling, wherein the dosing regimen comprises about 600 mg of dupilumab (marketed as DUPIXENT®) as a first dose, followed by two subsequent doses of about 300 mg of dupilumab about every three weeks thereafter. Other dosing regimens are within the scope of the disclosure and are described herein.

### Definitions

Unless defined otherwise, all technical and scientific terms used herein the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

### Antibody

The term “antibody,” as may be used herein, refers to immunoglobulins (IG) comprising four peptides (e.g., polypeptides, proteins.). The peptides of antibodies are referred to and termed “chains,” which are connected via disulfide bonds. The chains of an antibody are sub-divided into two heavy (H) chains and two light (L) chains. Each H chain comprises a heavy chain variable region (VH) and a heavy chain constant region (CH). The CH is further comprised of three domains CH1-CH3. Similar to the H chain, each L chain comprises an L chain variable region (VL) and a L chain constant region (CL). However, dissimilarly from the CH, the CL comprises a single domain.

The VH and VL regions of the H and L chains, respectively, are further sub-divided. These regions are known to be highly influential to the specificity of the antibody due to the fact they contain exceedingly high levels of variability (e.g., hypervariability). These elements of hypervariable regions, are referred to as “complementarity determining regions (CDRs).” These CDRs are supported by a scaffold portion of the variable region of the antibody (e.g., VH, VL), known as framework regions. These framework regions are known to be less variable than the CDRs (e.g., more conserved among and across antibodies).

In this specification “antibody” includes a fragment or derivative of an antibody, or a synthetic antibody or synthetic antibody fragment. Antibodies may be provided in isolated or purified form. Antibodies may be formulated as a pharmaceutical composition or medicament.

In view of today's techniques in relation to monoclonal antibody technology, antibodies can be prepared to most antigens, e.g., an IL-4 or IL-4 receptor. The antigen-binding portion may be a part of an antibody (for example a Fab fragment) or a synthetic antibody fragment (for example a single chain Fv fragment [ScFv]). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimeric antibodies are discussed by Neuberger et al (1988, 8th International Biotechnology Symposium Part 2, 792-799).

Monoclonal antibodies (mAbs) are useful in the methods of the invention and are a homogenous population of antibodies specifically targeting a single epitope on an antigen.

Polyclonal antibodies are useful in the methods of the invention. Monospecific polyclonal antibodies are preferred. Suitable polyclonal antibodies can be prepared using methods well known in the art.

Antigen binding fragments of antibodies, such as Fab and Fab₂ fragments may also be used/provided as can genetically engineered antibodies and antibody fragments. The variable heavy (V_{H}) and variable light (V_{L}) domains of the antibody are involved in antigen recognition, a fact first recognized by early protease digestion experiments. Further confirmation was found by “humanization” of rodent antibodies. Variable domains of rodent origin may be fused to constant domains of human origin such that the resultant antibody retains the antigenic specificity of the rodent parented antibody (Morrison et al (1984) Proc. Natl. Acad. Sd. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sd. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

The term “ScFv molecules” refers to molecules wherein the V_{H} and V_{L} partner domains are covalently linked, e.g. by a flexible oligopeptide.

Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. The term “bivalent” means that the said antibodies and F(ab′)₂ fragments have two antigen combining sites. In contrast, Fab, Fv, ScFv and dAb fragments are monovalent, having only one antigen combining site. Synthetic antibodies which bind to IL-11 or IL-11R may also be made using phage display technology as is well known in the art.

Antibodies may be produced by a process of affinity maturation in which a modified antibody is generated that has an improvement in the affinity of the antibody for antigen, compared to an unmodified parent antibody. Affinity-matured antibodies may be produced by procedures known in the art, e.g., Marks et al., Rio/Technology 10:779-783 (1992); Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):331 0-15 9 (1995); and Hawkins et al, J. Mol. Biol. 226:889-896 (1992).

Antibodies according to the present invention may exhibit specific binding to an antigen. In various embodiments, the disclosure provides a combination anti-cancer treatment that combines an IL-4 signaling blocking agent (e.g. dupilumab) with a PD-(L)1 blocking agent (e.g., pembrolizumab). In some embodiments, the IL-4 signaling blocking agent is a human monoclonal antibody to the interleukin-4 (IL-4) receptor alpha subunit which disrupts IL-4 signaling through receptors for both IL-4 and IL-13, two cytokines known to be classic Th-2 polarizing cytokines. In still other embodiments, the PD-(L)1 blocking agent can be that specifically binds to IL-4 or IL-4 receptor (IL-4R) and/or to PD-1 or PD-1R (immune checkpoint blocking agent, such as pembrolizumab. An antibody that specifically binds to a target molecule preferably binds the target with greater affinity, and/or with greater duration than it binds to other targets. In one embodiment, the extent of binding of an antibody to an unrelated target is less than about 10% of the binding of the antibody to the target as measured, e.g., by ELISA, or by a radioimmunoassay (RIA). Alternatively, the binding specificity may be reflected in terms of binding affinity where the antibody binds to IL-4 or IL-4R with a K_{D} that is at least 0.1 order of magnitude (i.e. 0.1×10ⁿ, where n is an integer representing the order of magnitude) greater than the K_{D} of the antibody towards another target molecule.

Antibodies may be detectably labelled or, at least, capable of detection. Such antibodies being useful for both in vivo (e.g. imaging methods) and in vitro (e.g. assay methods) applications For example, the antibody may be labelled with a radioactive atom or a colored molecule or a fluorescent molecule or a molecule which can be readily detected in any other way. Suitable detectable molecules include fluorescent proteins, luciferase, enzyme substrates, and radiolabels. The binding moiety may be directly labelled with a detectable label or it may be indirectly labelled. For example, the binding moiety may be an unlabeled antibody which can be detected by another antibody which is itself labelled. Alternatively, the second antibody may have bound to it biotin and binding of labelled streptavidin to the biotin is used to indirectly label the first antibody.

Aspects of the present invention include bi-specific antibodies, e.g. composed of two different fragments of two different antibodies, such that the bi-specific antibody binds two types of antigen. One of the antigens is IL-4 or IL-4R, the bi-specific antibody comprising a fragment as described herein that binds to IL-4 or IL-4R. The antibody may contain a different fragment having affinity for a second antigen, which may be any desired antigen. Techniques for the preparation of bi-specific antibodies are well known in the art, e.g. see Mueller, D et al., (2010 Biodrugs 24 (2): 89-98), Wozniak-Knopp G et al., (2010 Protein Eng Des 23 (4): 289-297. Baeuerle, P A et al., (2009 Cancer Res 69 (12): 4941-4944).

In some embodiments, the bispecific antibody is provided as a fusion protein of two single-chain variable fragments (scFV) format, comprising a V_{H} and V_{L} of a IL-4 or IL-4R binding antibody or antibody fragment, and a V_{H} and V_{L} of an another antibody or antibody fragment.

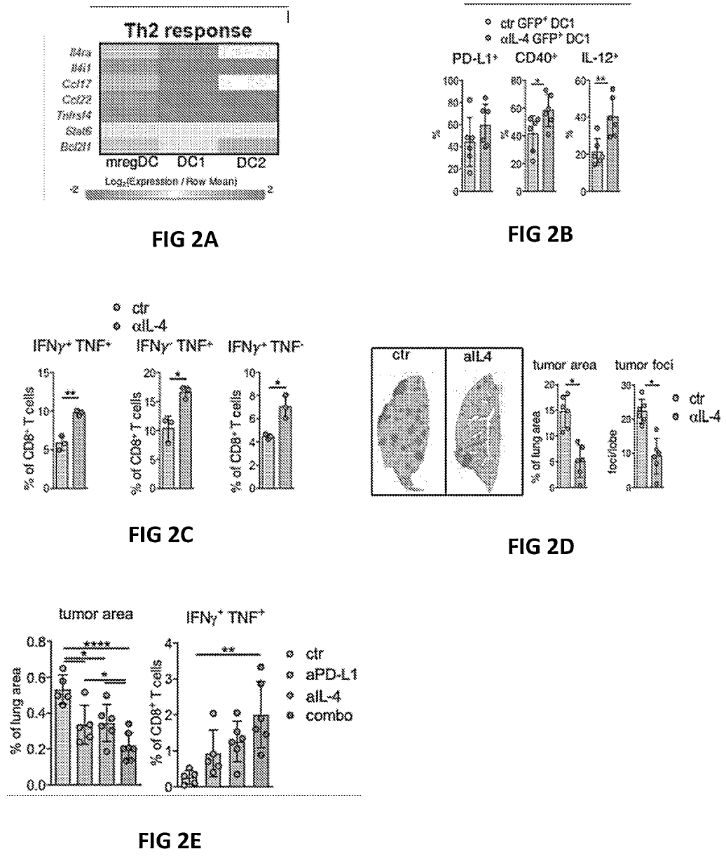

Bispecific antibodies and bispecific antigen binding fragments may be provided in any suitable format, such as those formats described in Kontermann MAbs 2012, 4(2): 182-197, which is hereby incorporated by reference in its entirety. For example, a bispecific antibody or bispecific antigen binding fragment may be a bispecific antibody conjugate (e.g. an IgG2, F(ab′)₂ or CovX-Body), a bispecific IgG or IgG-like molecule (e.g. an IgG, scFv₄-Ig, IgG-scFv, scFv-IgG, DVD-Ig, IgG-sVD, sVD-IgG, 2 in 1-IgG, mAb², or Tandemab common LC), an asymmetric bispecific IgG or IgG-like molecule (e.g. a kih IgG, kih IgG common LC, CrossMab, kih IgG-scFab, mAb-Fv, charge pair or SEED-body), a small bispecific antibody molecule (e.g. a Diabody (Db), dsDb, DART, scDb, tandAbs, tandem scFv (taFv), tandem dAb/VHH, triple body, triple head, Fab-scFv, or F(ab′)₂-scFv₂), a bispecific Fc and C_{H}3 fusion protein (e.g. a taFv-Fc, Di-diabody, scDb-C_{H}3, scFv-Fc-scFv, HCAb-VHH, scFv-kih-Fc, or scFv-kih-C_{H}3), or a bispecific fusion protein (e.g. a scFv₂-albumin, scDb-albumin, taFv-toxin, DNL-Fab₃, DNL-Fab₄-IgG, DNL-Fab₄-IgG-cytokine₂). See in particular FIG. 2 of Kontermann MAbs 2012, 4(2): 182-19.

Methods for producing bispecific antibodies include chemically crosslinking antibodies or antibody fragments, e.g., with reducible di-sulfide or non-reducible thioether bonds, for example as described in Segal and Bast, 2001. Production of Bispecific Antibodies. Current Protocols in Immunology. 14:IV:2.13:2.13.1-2.13.16, which is hereby incorporated by reference in its entirety. For example, N-succinimidyl-3-(−2-pyridyldithio)-propionate (SPDP) can be used to chemically crosslink e.g., Fab fragments via hinge region SH— groups, to create disulfide-linked bispecific F(ab)₂ heterodimers.

Other methods for producing bispecific antibodies include fusing antibody-producing hybridomas e.g. with polyethylene glycol, to produce a quadroma cell capable of secreting bispecific antibody, for example as described in D. M. and Bast, B. J. 2001. Production of Bispecific Antibodies. Current Protocols in Immunology. 14:IV:2.13:2.13.1-2.13.16.

Bispecific antibodies and bispecific antigen binding fragments can also be produced recombinantly, by expression from a nucleic acid construct encoding polypeptides for the antigen binding molecules, for example as described in Antibody Engineering: Methods and Protocols, Second Edition (Humana Press, 2012), at Chapter 40: Production of Bispecific Antibodies: Diabodies and Tandem scFv (Hornig and Färber-Schwarz), or French, How to make bispecific antibodies, Methods Mol. Med. 2000; 40:333-339.

For example, a DNA construct encoding the light and heavy chain variable domains for the two antigen binding domains (i.e. the light and heavy chain variable domains for the antigen binding domain capable of binding IL-4 or IL-4R, and the light and heavy chain variable domains for the antigen binding domain capable of binding to another target protein), and including sequences encoding a suitable linker or dimerization domain between the antigen binding domains can be prepared by molecular cloning techniques. Recombinant bispecific antibody can thereafter be produced by expression (e.g., in vitro) of the construct in a suitable host cell (e.g., a mammalian host cell), and expressed recombinant bispecific antibody can then optionally be purified.

### Anti-IL-4 Signaling Antibody

The term “anti-IL-4 signaling antibody,” as may be used herein, refers to an antibody that directly or indirectly blocks or reduces IL-4 signaling. In certain embodiments, an anti-IL-4 signaling antibody can be an antibody that specifically binds to IL-4, thereby blocking or reducing the ability of IL-4 from binding the IL-4 receptor. In certain other embodiments, an anti-IL-4 signaling antibody can be an antibody that specifically binds to the IL-4 receptor, thereby blocking or reducing the ability of IL-4 from binding to the IL-4 receptor.

### Anti-IL-4 Antibody

The term “anti-IL-4 antibody,” as may be used herein, refers to an antibody that specifically binds to IL-4 and blocks the interaction between IL-4 and IL-4R, thereby blocking or reducing IL-4 signaling. Antibodies that specifically bind to IL-4 are well-known in the art.

In other embodiments, the anti-IL-4 antibody can be any known (published and/or commercially available) anti-IL-4 antibody. Such antibodies can be obtained, for example, from commercial suppliers, including INVITROGEN, PROTEINTECH, and ENZO LIFE SCIENCES.

In still other embodiments, anti-IL-4 antibodies can be prepared by methods well-known in art and discussed below.

### Anti-IL-4 Receptor Antibody

The term “anti-IL-4 receptor antibody,” as may be used herein, refers to an antibody that specifically binds to IL-4 receptor and blocks the interaction between IL-4 and IL-4R, thereby blocking or reducing IL-4 signaling. Antibodies that specifically bind to IL-4 receptor are well-known in the art.

In one embodiment, the anti-IL-4 receptor antibody can be dupilumab, which is marketed under the name DUPIXENT®. Dupilumab binds to the alpha subunit of the interleukin-4 receptor (IL-4Rα), making it a receptor antagonist. Through blockade of IL-4Rα, dupilumab modulates signaling of the IL-4 pathway.

In other embodiments, the anti-IL-4 receptor antibody can be any known (published and/or commercially available) anti-IL-4 receptor antibody. Such antibodies can be obtained, for example, from commercial suppliers, including INVITROGEN, PROTEINTECH, and ENZO LIFE SCIENCES.

In still other embodiments, anti-IL-4 antibodies can be prepared by methods well-known in art and discussed below.

### Antibody Fragment

The terms “antigen binding portion,” “antigen binding fragment,” and “antibody fragment,” as may be used interchangeably herein, refer to one, or more, portions (e.g., fragments) of an antibody that retain the ability to specifically bind to the antigen of the antibody of which they a portion or fragment (e.g., IL-4, PD-1, PDL-1). It is well known in the art and readily appreciated by the skilled artisan that a peptide of an antibody (e.g., less than the entire protein sequence and structure of a complete antibody (e.g., portion, fragment of full-length antibody)) can bind and perform the function of a full-length antibody. For example, without limitation, known examples of antibody fragments within the term include, Fab fragments, monovalent fragments, F(ab′)2 fragments, bivalent fragments, Fd fragments, Fv fragments, dAb fragments, and CDRs. Linkers (e.g., synthetic linkers) make recombinant versions of these arrangements available to the skilled artisan, allowing for domains of antibodies encoded by separate genes to be assembled into single chains.

### Blocking Antibody

As used herein, the term “blocking antibody” or equivalently, “neutralizing antibody,” refers to an antibody whose binding to its intended antigen (e.g., IL-4) results inhibition of the biological activity of the antigen (e.g., IL-4). Methods of measuring inhibition as stated above are well known in the art and readily appreciated by the skilled artisan. For example, the use and subsequent measurement of tagged or modified antibodies may be used to quantify inhibition.

### Combination Therapy

The terms “combination therapy” and “polytherapy,” as may be used interchangeably herein, generally refer to the application (e.g., use) of multiple therapies (e.g., modes of treatment, such as drugs, therapeutics, or interventions) to treat a common (e.g., single) disease or disorder, or symptoms related thereto. Often, a disease or disorder is targeted directly by multiple therapies (e.g., at least two), but in instances, one therapy may treat a symptom (or more than one symptom), while another therapy may treat the underlying disease or disorder causing the symptoms. However, multiple therapies may treat the disease or disorder as well as multiple therapies treating the symptoms thereof. In some embodiments, a combination therapy comprises at least two (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more) different treatments which target a disease or disorder, or symptoms related thereto. In some embodiments, a combination therapy comprises at least two (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more) different treatments, of which at least one treats the disease or disorder, while at least one other treats the symptoms related thereto. In some embodiments, a combination therapy comprises at least two (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more) different treatments which target a disease or disorder, or symptoms related thereto, wherein at least two treat the disease or disorder (e.g., the cause of the symptoms related thereto). The skilled artisan will readily appreciate that such term, combination therapy, shall also include the use of adjuvants. The term “adjuvant.” as may be used herein, refers to any therapy or treatment (e.g., composition, drug, or method based) which is used as an adjunct to the primary or initial therapy or treatment. Adjuvants may be administered concurrently (e.g., at the same time, simultaneously) with the primary or initial treatment or shortly after the administration of the primary or initial treatment. In some, but not all, cases an adjuvant modulates (e.g., increases, decreases) the effect of the primary or initial treatment. In some, but not all, cases an adjuvant is used to modulate (e.g., increase, decrease) a side effect of the primary or initial treatment. In some, but not all, cases an adjuvant is used to prepare (e.g., condition) a subject in anticipation of the primary or initial treatment or aid in the primary or initial treatment's effects or sustain or aid in the recovery of the subject after the primary or initial treatment. A “neoadjuvant,” as may be used herein, refers to an adjuvant which is administered prior to the primary treatment. Neoadjuvants are often given to improve the likelihood that the overall treatment or therapy will yield a favorable outcome. This may be accomplished in a variety of ways, for example, by improving the efficacy, tolerability (e.g., reducing the toxicity or side-effects), targeting (improving targeting, or reducing off-target effects) of the primary treatment.

In some embodiments, a combination therapy comprises administering to a subject at least one anti-IL-4 signaling antibody and at least one anti-programmed cell death protein 1 (anti-PD1) antibody. In some embodiments, a combination therapy comprises administering to a subject at least one anti-IL-4 signaling antibody and at least one anti-programmed death ligand 1 (anti-PD-L1) antibody. In some embodiments, an anti-IL-4 antibody is dupilumab (marketed as DUPIXENT®).

In other embodiments, a combination therapy comprises administering to a subject at least one anti-IL-4 signaling antibody and at least one immune checkpoint inhibitor, such as, but not limited to (1) ipilimumab (marketed as YERVOY®), an anti-CTLA-4 antibody, (2) tremelimumab, an anti-CTLA-4 antibody. (3) nivolumab (marketed as OPDIVO®), an anti-PD-1 antibody, (4) pembrolizumab (marketed as KEYTRUDA®), an anti-PD-1 antibody, (5) pidilizumab, and anti-PD-1 antibody, (6) MEDI0680, an anti-PD-1 antibody, (7) atezolizumab, an anti-PD-L1 antibody (marketed as TECETRIQ®), (8) avelumab, an anti-PD-L1 antibody (marketed as BAVENCIO®), (9) durvalumab, an anti-PD-L1 antibody (marketed as IMFINZI®), or (10) cemiplimab, an anti-PD-1 antibody (marketed as LIBTAYO®).

In some embodiments, an anti-IL-4 signaling antibody is dupilumab (marketed as DUPIXENT®).

The combination therapy may include the administration of an anti-IL4 signaling antibody (e.g., dupilumab) and an immune checkpoint inhibitor (e.g., anti-PD-(L)1 antibody) at the same time, wherein one or more doses of each are administered at the same time. In other embodiments, the combination therapy may include the administration of an anti-IL4 signaling antibody (e.g., dupilumab) and an immune checkpoint inhibitor (e.g., anti-PD-(L)1 antibody) at different times, wherein one or more doses of each are administered at different times.

### Complementarity Determining Regions

The terms “complementarity determining regions” and “CDRs.” as may be used interchangeably herein, refer to regions of the VH and VL which contain hypervariable regions. As noted above, these CDRs are supported by and framed by the framework regions (FRs), which regions are interspersed with the CDRs of the antibody. Each VH and VL is composed of three CDRs (CDR1-CDR3) and four FRs (FR1-FR4), arranged from amino-terminus (N-terminus) to carboxy-terminus (C-terminus): FR1, CDR1, FR2, CDR2, FR3, CDR3, and FR4.

### Dupilumab (DUPIXENT®)

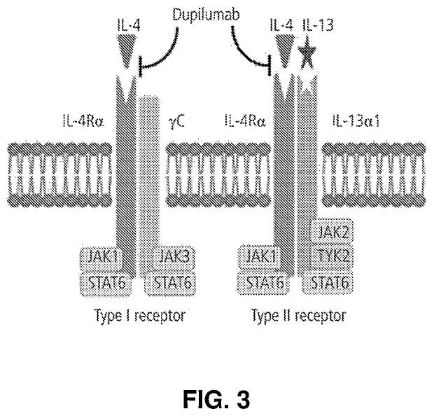

Dupilumab (marketed as DUPIXENT® by Regeneron Pharmaceuticals) is a monoclonal antibody dual-inhibitor of IL-4 and IL-13 signaling and as of this filing is approved for three indications (atopic dermatitis, asthma, and chronic rhinosinusitis with nasal polyposis) in the United States by the Food and Drug Administration (FDA). Dupilumab inhibits the biological effects of the cytokines IL-4 and IL-13, which are key drivers in the TH₂ response. Dupilumab is a fully human IgG4 monoclonal antibody directed against IL-4Rα, which inhibits IL-4/IL-13 signaling and thus downregulates type-2 immunity. There are 2 types of IL-4 receptors: the type 1 receptor, which is composed of the IL-4 chain (IL-4Rα) and a γ chain (γC), and the type 2 receptor, which is composed of the IL-4Rα chain and the α1 chain of the IL-13 receptor (IL-13Rα1) (see FIG. 3). These receptors are present in the surface of a large number of cells involved in the pathophysiology of TH₂ allergic responses, which include B lymphocytes, eosinophils, dendritic cells, monocytes/macrophages, basophils, keratinocytes, bronchial epithelial cells, endothelial cells, fibroblasts, and airway smooth muscle cells. The type 1 receptor can be activated by IL-4 and the type 2 receptor can be activated by both IL-4 and IL-13 (FIG. 3). Ligand binding to these receptors activates a signal transduction cascade that mainly leads to the modulation of expression of genes involved in IgE class switching. TH₂ cell differentiation, and M2 macrophage polarization. Evidence from basic research and a very large body of evidence from clinical trials supports the model by which dupilumab binding to IL-4 receptors inhibits their activation by IL-4 and IL-13, thus blocking the signaling pathways involved in the development and progression of allergic responses. See Sastre J and Davila I, “Dupilumab: A New Paradigm for the Treatment of Allergic Diseases,” J Investig Allergol Clin Immunol 2018; Vol. 28(3): 139-150.

### Dendritic Cells

Dendritic cells are a type of antigen-presenting cell (APC) that form an important role in the adaptive immune system. The main function of dendritic cells is to present antigens and the cells are therefore sometimes referred to as “professional” APCs. Dendritic cells also contribute to the function of B cells and help maintain their immune memory. Dendritic producing cytokines and other factors that promote B cell activation and differentiation. After an initial antibody response has occurred due to an invading body, dendritic cells found in the germinal center of lymph nodes seem to contribute to B cell memory by forming numerous antibody-antigen complexes. This is to provide a long-lasting source of antigen that the B cells can take up themselves and present to T cells. Dendritic cells are found in tissue that has contact with the outside environment such as the over the skin (present as Langerhans cells) and in the linings of the nose, lungs, stomach and intestines. Immature forms are also found in the blood. Once activated, dendritic cells move to the lymph tissue to interact with to interact with T cells and B cells and help shape the adaptive immune response.

Dendritic cells (DCs), named for their probing, ‘tree-like’ or dendritic shapes, are responsible for the initiation of adaptive immune responses and hence function as the ‘sentinels’ of the immune system. Paul Langerhans first described DCs in human skin in 1868 but thought they were cutaneous nerve cells. DCs are bone marrow (BM)-derived leukocytes and are the most potent type of antigen-presenting cells. DCs are specialized to capture and process antigens, converting proteins to peptides that are presented on major histocompatibility complex (MHC) molecules recognized by T cells. DCs are heterogeneous, e.g., myeloid and plasmacytoid DCs; although all DCs are capable of antigen uptake, processing and presentation to naive T cells, the DC subtypes have distinct markers and differ in location, migratory pathways, detailed immunological function and dependence on infections or inflammatory stimuli for their generation. During the development of an adaptive immune response, the phenotype and function of DCs play an extremely important role in initiating tolerance, memory, and polarized T-helper 1 (Th1), Th2 and Th17 differentiation.

Dendritic cells are derived from the bone-marrow cells, arising from lympho-myeloid haematopoiesis that form an essential interface between the innate sensing of pathogens and the activation of adaptive immunity. This task requires a wide range of mechanisms and responses, which are divided between three major DC subsets: plasmacytoid DC (pDC), myeloid/conventional DC1 (cDC1 or DC1) and myeloid/conventional DC2 (cDC2 or DC2).

The present inventors have discovered a new subset of DCs referred to herein as “mature DCs enriched in immunoregulatory molecules” or “mregDCs”.

### Effective Amount

An “effective amount” of a therapeutic agent described herein (e.g., an antibody) refers to an amount sufficient to elicit the desired biological response. An effective amount of a therapeutic agent described herein may vary depending on such factors as the desired biological endpoint, the pharmacokinetics of a therapeutic agent, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

In some embodiments, “an effective amount” refers to the amount of each active agent (e.g., anti-IL-4 antibodies, anti-PD-1 antibodies, anti-PDL-1 antibodies, combinations thereof) required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

### Epitope

The term “epitope,” as may be used herein, refers to the portion of an antigen that is recognized by an antigen binding site on an antibody. On the antibody, the antigen binding site resides in the variable regions (e.g., V_{H}, V_{L}) and is referred to as the paratope. Epitope-paratope relationships and combinations are not limited to a 1:1 ratio. In other words, an antigen may be recognized by multiple antibodies (e.g., have more than one epitope). These epitopes may be either conformational or linear. Conformational epitopes result from the positioning of non-consecutive amino acids in the antigen near or adjacent to one another such that they are recognized together. Linear epitopes result from the recognition of sequential or consecutive amino acids in the peptide sequence of an antigen.

### Immune Checkpoint

As used herein, the term “immune checkpoint” refer to regulators of the immune system that affect immune system self-tolerance and help prevent the immune system from attacking cells indiscriminately. For example, when the immune system is attacking pathogens, immune checkpoint molecules can protect normal tissues from being damaged by the immune response against the pathogens. Some cancer cells have leveraged immune checkpoints to escape from immune attack by dysregulating immune checkpoint proteins, which include, but are not limited to cytotoxic T-lymphocyte-associated protein 4 (CTLA-4), programmed cell death protein 1 (PD-1), programmed cell death ligand 1 (PD-L1), and programmed cell death ligand 2 (PD-L2). Immune checkpoints are targets for cancer immunotherapy due to their potential for use in multiple types of cancers. Currently approved checkpoint inhibitors block CTLA4 and PD-1 and PD-L1.

Other checkpoints can include, but are not limited to:

A2AR: The Adenosine A2A receptor is regarded as an important checkpoint in cancer therapy because adenosine in the immune microenvironment, leading to the activation of the A2a receptor, is negative immune feedback loop and the tumor microenvironment has relatively high concentrations of adenosine.

B7-H3: also called CD276, was originally understood to be a co-stimulatory molecule but is now regarded as co-inhibitory.

B7-H4: also called VTCN1, is expressed by tumor cells and tumor-associated macrophages and plays a role in tumor escape.

BTLA: also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA.

IDO: short for Indoleamine 2,3-dioxygenase, is a tryptophan catabolic enzyme with immune-inhibitory properties. Another molecule is TDO, tryptophan 2,3-dioxygenase. IDO is known to suppress T and NK cells, generate and activate Tregs and myeloid-derived suppressor cells, and promote tumor angiogenesis.

KIR: short for Killer-cell Immunoglobulin-like Receptor, is a receptor for MHC Class I molecules on Natural Killer cells.

LAG3: short for Lymphocyte Activation Gene-3, works to suppress an immune response by action to Tregs as well as direct effects on CD8+ T cells.

NOX2: short for nicotinamide adenine dinucleotide phosphate NADPH oxidase isoform 2, is an enzyme of myeloid cells that generates immunosuppressive reactive oxygen species. Genetic and pharmacological inhibition of NOX2 in myeloid cells improves anti-tumor functions of adjacent NK cells and T cells and also triggers autoimmunity in humans and experimental animals.

TIM-3: short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9.

VISTA: Short for V-domain Ig suppressor of T cell activation, VISTA is primarily expressed on hematopoietic cells so that consistent expression of VISTA on leukocytes within tumors may allow VISTA blockade to be effective across a broad range of solid tumors.

SIGLEC7 (Sialic acid-binding immunoglobulin-type lectin 7, also designated as CD328) and SIGLEC9 (Sialic acid-binding immunoglobulin-type lectin 9, also designated as CD329) are proteins found on the surface of various immune cells, including natural killer cells and macrophages (SIGLEC7) and neutrophils, macrophages, dendritic cells and activated T-cells (SIGLEC9). SIGLECs 7 and 9 suppress the immune function of these cells by binding to terminal sialic acid on glycans that cover the surface of cells.

### Immune Checkpoint Inhibitor

As used herein, the term “immune checkpoint inhibitor” (“ICI”) or equivalently, “checkpoint inhibitor,” refers to an agent which targets immune checkpoints. Immune system checkpoints operate to modulate (e.g., increase, decrease, start, stop, etc.) immunologic activity (e.g., immune response to an immunologic stimulus. In the case where a disorder (e.g., indication, disease, for example cancer) which exploits immune checkpoints (i.e., stimulates them as to decrease the host immune response to the disorder (e.g., immunologic stimulus)), inhibitors (e.g., checkpoint inhibitors) may be advantageously used to inhibit the effect of the checkpoints, thus decreasing the inhibitory effect of the checkpoint (i.e., increasing or restoring the immunologic response to the disorder). Non-limiting examples of checkpoint inhibitors include, but are not limited to (1) ipilimumab (marketed as YERVOY®), an anti-CTLA-4 antibody, (2) tremelimumab, an anti-CTLA-4 antibody. (3) nivolumab (marketed as OPDIVO®), an anti-PD-1 antibody, (4) pembrolizumab (marketed as KEYTRUDA®), an anti-PD-1 antibody, (5) pidilizumab, and anti-PD-1 antibody, (6) MEDI0680, an anti-PD-1 antibody, (7) atezolizumab, an anti-PD-L1 antibody, (8) avelumab, an anti-PD-L1 antibody, (9) durvalumab, an anti-PD-L1 antibody, and (10) cemiplimab, an anti-PD-1 antibody.

### IL-4

The terms “interleukin-4” and “IL-4,” as may be used interchangeably herein, refer to a cytokine that specifically binds to IL-4 receptor (IL-4R). In some embodiments, IL-4 is a mammalian IL-4 cytokine. In some embodiments, IL-4 is a human IL-4 cytokine. Without being bound by theory, IL-4 has many biological roles, including the stimulation of activated B-cell and T-cell proliferation, and the differentiation of B cells into plasma cells. It is a key regulator in humoral and adaptive immunity. IL-4 induces B-cell class switching to IgE, and up-regulates MHC class II production. IL-4 is recognized as playing a pivotal role in shaping the nature of immune responses.

Interleukin-4 plays a critical role in initiating and regulating Th2-type immune responses. In mice, IL-4 is a 14-19 kDa glycoprotein localized on chromosome 11, together with the genes for IL-5 and IL-13. During the innate immune response, evidence suggests that early IL-4-producers include basophils, mast cells, eosinophils, natural killer (NK) T cells, and innate-like skin keratinocytes. T and B lymphocytes orchestrating adaptive immunity, specifically CD4+ Th2 cells, B effector 2 (Be2) B cells, and γ/δ T cells, also secrete IL-4. Apart from regulating the differentiation of Th2 cells, IL-4 also controls immunoglobulin class switching in activated B cells, specifying human B cells to switch to the expression of IgE and IgG4, while in mice, to IgE and IgG1, with the concomitant suppression of IgM, IgG2a, and IgG2b. Moreover, alternatively activated macrophages are activated by IL-4 signaling through the IL-4Rα chain. Importantly, IL-4 inhibits inducible nitric oxide synthase (iNOS) expression thereby inhibiting IFN-γ-induced classically activated macrophages and induction of a type 1 response. As a whole, IL-4 counter-regulates the expression of IFN-γ and increases the expression of MHC II molecules, co-stimulatory molecules CD80 and CD86, and the IL-4 receptor. Reports have also indicated that dendritic cells (DCs) can respond to IL-4 in vivo and in vitro and become alternatively activated, in a manner similar to that described for alternatively activated macrophages, by upregulating multiple alternative activation markers such as mannose receptor and RELM-α. Moreover, although IL-4 has been shown to be the primary inducer of Th2 responses, studies have reported IL-4-independent Th2 differentiation, Th2 cytokine production, IL-4Rα signaling, and STAT6 regulation.

### IL-4 Receptor

The term “interleukin-4 receptor,” or equivalently, IL-4R or IL-4Rα, refers to the receptor of IL-4 and is a type I cytokine receptor. In some embodiments, IL-4R is a mammalian IL-4R. In some embodiments, IL-4R is a human IL-4R. IL-4R, a type I transmembrane protein, can bind interleukin-4 and interleukin-13 to regulate IgE antibody production in B cells. Among T cells, the encoded protein also can bind interleukin-4 to promote differentiation of Th2 cells. Allelic variations in this gene have been associated with atopy, a condition that can manifest itself as allergic rhinitis, sinusitis, asthma, or eczema. Two transcript variants encoding different isoforms, a membrane-bound and a soluble form, have been found for this gene. Interactions of IL-4 with TNFα promote structural changes to vascular endothelial cells, thus playing an important role in tissue inflammation.

The binding of IL-4 or IL-13 to the IL-4 receptor on the surface of macrophages results in the alternative activation of those macrophages. Alternatively activated macrophages (AAMΦ) downregulate inflammatory mediators such as IFNγ during immune responses, particularly with regards to helminth infections.

### Linker

The term “linker,” as used herein, refers to a chemical moiety linking two molecules or moieties. Typically, the linker is positioned between, or flanked by, two groups, molecules, or other moieties and connected to each one via a covalent bond, thus connecting the two. In some embodiments, the linker comprises an amino acid or a plurality of amino acids (e.g., a peptide or protein). In some embodiments, the linker comprises a nucleotide (e.g., DNA or RNA) or a plurality of nucleotides (e.g., a nucleic acid). In some embodiments, the linker is an organic molecule, functional group, group, polymer, or other chemical moiety. In some embodiments, the linker is a cleavable linker, e.g., the linker comprises a bond that can be cleaved upon exposure to, for example, UV light or a hydrolytic enzyme, such as a protease or esterase. In some embodiments, the linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids). In other embodiments, the linker is a chemical bond (e.g., a covalent bond, amide bond, disulfide bond, ester bond, carbon-carbon bond, carbon heteroatom bond).

### Pembrolizumab (KEYTRUDA®)

Pembrolizumab is an anti-PD-1 antibody and immune checkpoint inhibitor approved in the U.S. in 2014 as KEYTRUDA® for the treatment of a variety of cancers, including melanoma, NSCLC, and mesothelioma, among others. Administration of pembrolizumab acts to block PD-(L)1 checkpoint inhibitor signaling, thereby activating the immune system to better kill cancer cells. Specifically, PD-1 is a protein on the surface of activated T cells. If another molecule, called programmed cell death 1 ligand 1 or programmed cell death 1 ligand 2 (PD-L1 or PD-L2) binds to PD-1, the initially-activated T cell becomes inactive. This is one way that the body regulates the immune system, to avoid an overreaction. Many cancer cells make PD-L1, which inhibits T cells from attacking the tumor. Pembrolizumab blocks PD-L1 from binding to PD-1, allowing the T cell to work against the cancer cells.

### Percent Identity

The terms “percent identity.” “sequence identity.” “% identity.” “% sequence identity.” and % identical,” as they may be interchangeably used herein, refer to a quantitative measurement of the similarity between two sequences (e.g., nucleic acid or amino acid). The percent identity of genomic DNA sequence, intron and exon sequence, and amino acid sequence between humans and other species varies by species type, with chimpanzee having the highest percent identity with humans of all species in each category.

Calculation of the percent identity of two nucleic acid sequences, for example, can be performed by aligning the two sequences for optimal comparison purposes (e.g., gaps can be introduced in one or both of a first and second nucleic acid sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Atschul, S. F. et al., J. Molec. Biol., 215, 403 (1990)).

When a percent identity is stated, or a range thereof (e.g., at least, more than, etc.), unless otherwise specified, the endpoints shall be inclusive and the range (e.g., at least 70% identity) shall include all ranges within the cited range (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) and all increments thereof (e.g., tenths of a percent (e.g., 0.1%), hundredths of a percent (e.g., 0.01%), etc.).

### Subject

The term “subject,” as used herein, refers to any organism in need of treatment or diagnosis using the subject matter herein (e.g., methods of treating a subject using IL-4 antibodies). For example, without limitation, subjects may include mammals and non-mammals. In some embodiments, a subject is mammalian. In some embodiments, a subject is non-mammalian. As used herein, a “mammal,” refers to any animal constituting the class Mammalia (e.g., a human, mouse, rat, cat, dog, sheep, rabbit, horse, cow, goat, pig, guinea pig, hamster, chicken, turkey, or a non-human primate (e.g., Marmoset, Macaque)). In some embodiments, subject is a human.

### Treatment

The terms “treatment,” “treat,” and “treating,” as may be used interchangeably herein, refer to partially or completely alleviating, ameliorating, relieving, delaying onset of, inhibiting progression of, reducing severity of, and/or reducing incidence of one or more symptoms or features of a particular indication, disease, disorder, condition, and/or symptom thereof. In some embodiments, the treatment refers to a clinical intervention. In some embodiments, treatment may be administered after one or more symptoms have developed and/or after a disease has been diagnosed. In other embodiments, treatment may be administered in the absence of symptoms (e.g., to prevent or delay onset of a symptom or inhibit onset or progression of a disease). For example, treatment may be administered to a susceptible individual (e.g., subject) prior to the onset of symptoms (e.g., in light of a history of symptoms and/or in light of genetic or other susceptibility factors). In some embodiments, the treatment is used and/or administered as a prophylaxis. Treatment may also be continued after symptoms have resolved, for example, to prevent or delay their recurrence.

Also, within the scope of the term treatment as described hereinabove, is a treatment that includes the administration of at least two therapeutic agents. This may be referred to as co-treatment or co-therapy. In some embodiments, a co-treatment comprises administering to a subject at least one anti-IL-4 signaling antibody and at least one anti-programmed cell death protein 1 (anti-PD1) antibody. In some embodiments, a co-treatment therapy comprises administering to a subject at least one anti-IL-4 signaling antibody and at least one anti-programmed death ligand 1 (anti-PD-L1) antibody. In some embodiments, an anti-IL-4 antibody is dupilumab (marketed as DUPIXENT®).

In other embodiments, a co-treatment comprises administering to a subject at least one anti-IL-4 signaling antibody and at least one immune checkpoint inhibitor, such as, but not limited to (1) ipilimumab (marketed as YERVOY®), an anti-CTLA-4 antibody, (2) tremelimumab, an anti-CTLA-4 antibody, (3) nivolumab (marketed as OPDIVO®), an anti-PD-1 antibody, (4) pembrolizumab (marketed as KEYTRUDA®), an anti-PD-1 antibody, (5) pidilizumab, and anti-PD-1 antibody, (6) MEDI0680, an anti-PD-1 antibody, (7) atezolizumab, an anti-PD-L1 antibody (marketed as TECETRIQ®), (8) avelumab, an anti-PD-L1 antibody (marketed as BAVENCIO®), (9) durvalumab, an anti-PD-L1 antibody (marketed as IMFINZI®), or (10) cemiplimab, an anti-PD-1 antibody (marketed as LIBTAYO®).

In some embodiments, an anti-IL-4 signaling antibody is dupilumab (marketed as DUPIXENT®).

The combination therapy may include the administration of an anti-IL4 signaling antibody (e.g., dupilumab) and an immune checkpoint inhibitor (e.g., anti-PD-(L)1 antibody) at the same time, wherein one or more doses of each are administered at the same time. In other embodiments, the combination therapy may include the administration of an anti-IL4 signaling antibody (e.g., dupilumab) and an immune checkpoint inhibitor (e.g., anti-PD-(L)1 antibody) at different times, wherein one or more doses of each are administered at different times.

In certain embodiments, the disclosure relates to activity taken to inhibit the inhibition (e.g., counteract a reduced effect) of biological processes and/or interventions (e.g., clinical, administrations of therapeutics, etc.). For example, without limitation it should be appreciated, that the use of an intervention (e.g., treatment) to inhibit or counteract checkpoint blockades is embodied by the term as used herein. Since a blockade operates to reduce the activity of its target, inhibiting the activation of a blockade (e.g., inhibition of an inhibition) reduces the inhibition and increases (e.g., restores) the activity of the blockades target and facilitating treatment of the disorder.

In some embodiments, the administration of an IL-4 antibody is administered to a subject as a treatment. In some embodiments, the IL-4 antibody is DUPIXENT® (dupilumab).

Types of cancers that may be treated include, but are not limited to, melanoma, sarcoma, Hodgkin's disease, leukemia, multiple myeloma, and lymphoma, along with cancer of the lung, pancreas, ovary, uterus, kidney, bladder, prostate, breast, head and neck, gastric, colorectal, and squamous cell carcinoma.

### Wildtype

The terms “wildtype” and “native,” as may be used interchangeably herein, are terms of art understood by skilled artisans and mean the typical form of an item, organism, strain, gene, or characteristic as it occurs in nature as distinguished from engineered, mutant, or variant forms.

### Abbreviations

The instant specification may include reference to one or more of the following abbreviations, which are defined as follows:

The abbreviation “AE” refers to an Adverse Event.

The abbreviation “ALT” refers to Alanine Aminotransferase.

The abbreviation “aPTT” refers to Activated Partial Thromboplastin Time.

The abbreviation “AST” refers to Aspartate Aminotransferase.

The abbreviation “BUN” refers to Blood Urea Nitrogen.

The abbreviation “CBC” refers to Complete Blood Count.

The abbreviation “CMP” refers to Comprehensive Metabolic Panel.

The abbreviation “CR” refers to Complete Response.

The abbreviation “CRF” refers to Case Report Form.

The abbreviation “CT” refers to Computed Tomography.

The abbreviation CTCAE Common Terminology Criteria for Adverse Events

The abbreviation “ctDNA” refers to Circulating Tumor DNA.

The abbreviation “CyTOF” refers to Time-of-Flight Mass Cytometry.

The abbreviation “DLT” refers to Dose Limiting Toxicity.

The abbreviation “DSMC” refers to Data and Safety Monitoring Committee.

The abbreviation “ECOG” refers to Eastern Cooperative Oncology Group.

The abbreviation “GFR” refers to Glomerular Filtration Rate.

The abbreviation “HAART” refers to Highly Active Antiretroviral Therapy.

The abbreviation “HBV” refers to Hepatitis B Virus.

The abbreviation “HCV” refers to Hepatitis C Virus.

The abbreviation “HIMC” refers to Human Immune Monitoring Core.

The abbreviation “INR” refers to International Normalized Ratio.

The abbreviation “irAE” refers to Immune-Related Adverse Event.

The abbreviation “IRB” refers to Institutional Review Board.

The abbreviation “IV (or iv)” refers to Intravenously.

The abbreviation “NSCLC” refers to Non-Small Cell Lung Cancer.

The abbreviation “ORR” refers to Overall Response Rate.

The abbreviation “OS” refers to Overall Survival.

The abbreviation “PBMCs” refers to Peripheral Blood Mononuclear Cells.

The abbreviation “PD” refers to Progressive Disease.

The abbreviation “PFS” refers to Progression Free Survival.

The abbreviation “PR” refers to Partial Response.

The abbreviation “PT” refers to Prothrombin Time.

The abbreviation “SAE” refers to Serious Adverse Event.

The abbreviation “SD” refers to Stable Disease.

The abbreviation “SOC” refers to Standard of Care.

The abbreviation “TSH” refers to Thyroid Stimulating Hormone

The abbreviation “WBC” refers to White Blood Cells.

### A. Combination Therapy

In various embodiments, the present disclosure provides a combination therapy comprising administering to a subject at least one anti-IL-4 signaling antibody and at least one anti-programmed cell death protein 1 (anti-PD1) antibody. In some embodiments, a combination therapy comprises administering to a subject at least one anti-IL-4 signaling antibody and at least one anti-programmed death ligand 1 (anti-PD-L1) antibody. In some embodiments, an anti-IL-4 antibody is dupilumab (marketed as DUPIXENT®).

In other embodiments, a combination therapy disclosed herein comprises administering to a subject at least one anti-IL-4 signaling antibody and at least one immune checkpoint inhibitor, such as, but not limited to (1) ipilimumab (marketed as YERVOY®), an anti-CTLA-4 antibody, (2) tremelimumab, an anti-CTLA-4 antibody, (3) nivolumab (marketed as OPDIVO®), an anti-PD-1 antibody, (4) pembrolizumab (marketed as KEYTRUDA®), an anti-PD-1 antibody, (5) pidilizumab, and anti-PD-1 antibody, (6) MEDI0680, an anti-PD-1 antibody, (7) atezolizumab, an anti-PD-L1 antibody (marketed as TECETRIQ®), (8) avelumab, an anti-PD-L1 antibody (marketed as BAVENCIO®), (9) durvalumab, an anti-PD-L1 antibody (marketed as IMFINZI®), or (10) cemiplimab, an anti-PD-1 antibody (marketed as LIBTAYO®).

In some embodiments, an anti-IL-4 signaling antibody is dupilumab.

The combination therapy may include the administration of an anti-IL4 signaling antibody (e.g., dupilumab) and an immune checkpoint inhibitor (e.g., anti-PD-(L)1 antibody) at the same time, wherein one or more doses of each are administered at the same time. In other embodiments, the combination therapy may include the administration of an anti-IL4 signaling antibody (e.g., dupilumab) and an immune checkpoint inhibitor (e.g., anti-PD-(L)1 antibody) at different times, wherein one or more doses of each are administered at different times.

The present disclosure relates to the field of cancer therapy. Specifically, the present disclosure relates to methods of treating cancer involving activating anti-tumor immunity by administering inhibitors of IL4 signaling to a subject in need thereof, particularly in combination with an immune checkpoint inhibitor.

Accordingly, the present disclosure provides anti-IL4 antibodies, anti-IL4R antibodies, and immune checkpoint therapies, and compositions comprising same, for the treatment of cancer. In one embodiment, the disclosure provides methods and compositions for treating cancer with anti-IL4 antibodies or anti-IL4R antibodies to induce an increased T cell activation function therein. In another embodiment, the disclosure provides a combination therapy comprising both an immune checkpoint therapy co-administered with an antibody that blocks IL-4 signaling, wherein the IL-4 signaling blockade sensitizes the tumors (either directly or indirectly) to the immune checkpoint therapy.

In some aspects, the disclosure relates to uses of anti-IL-4 binding proteins (e.g., antibodies) to treat diseases or disorders. In some embodiments, the anti-IL-4 antibodies are capable of modulating a disease or disorder associated with IL-4 signaling. In some embodiments, the disease or disorder is associated with a checkpoint blockade. In some embodiments, the disease or disorder is cancer.

The methods of treatment are not limited to any particular cancer or tumor. Non-limiting examples of cancers include: bladder cancer, breast cancer, colon and rectal cancer, endometrial cancer, kidney or renal cell cancer, leukemia, lung cancer, melanoma, Non-Hodgkin lymphoma, pancreatic cancer, prostate cancer, ovarian cancer, stomach cancer, wasting disease, and thyroid cancer. Additional non-limiting examples of cancer include Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hanlartoma, inesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, soft tissue Ewing's sarcoma, soft tissue sarcoma, synovial sarcoma, malignant lymphoma (reticulum cell sarcoma), multiple myeloma, malignant giant cell tumor chordoma, desmoid-type fibromatosis, fibroblastic sarcoma, gastrointestinal stromal tumors, retroperitoneal sarcoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis defomians), meninges (meningioma, meningiosarcoma, gliomatosis), brain (astrocytoma, medulloblastoma, glioma, ependymoma, germinoma [pincaloma], glioblastoma multiform, oligodendroglioma, schwannoma, retinoblastoma, congenital tumors), spinal cord neurofibroma, meningioma, glioma, sarcoma); gynaecological sarcoma, Kaposi's sarcoma, peripheral never sheath tumor, Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, SertoliLeydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles, dysplastic nevi, lipoma, angioma, dermatofibroma, keloids, psoriasis; and Adrenal glands: neuroblastoma. In some embodiments, a cancer is cancer of the respiratory system. In some embodiments, a cancer is lung cancer. In some embodiments, a cancer is liver cancer.

In some aspects, the disclosure relates to methods of treating a subject having, suspected of having, or at risk of having a disease or disorder associated with IL-4 signaling. A subject having a disease or disorder associated with IL-4 signaling can be identified by routine medical examination, e.g., laboratory tests, organ functional tests, CT scans, or ultrasounds. A subject suspected of having any of such disorder might show one or more symptoms of the disorder. A subject at risk for the disorder can be a subject having one or more of the risk factors for that disorder. In some embodiments, the method comprises administering to the subject an effective amount of an anti-IL-4 antibody. In some embodiments, the subject has, is suspected of having, or is at risk of having cancer. A subject having a cancer can be identified by routine medical examination, e.g., laboratory tests, organ functional tests, CT scans, or ultrasounds. A subject suspected of having cancer might show one or more symptoms of the disorder. A subject at risk for cancer can be a subject having one or more of the risk factors for that disorder. In some embodiments, a cancer is cancer of the respiratory system. In some embodiments, a cancer is lung cancer. In some embodiments, a cancer is liver cancer.

### B. Immune Checkpoint Blockade Antibodies

In various aspects, the present disclosure involves a combination anti-cancer therapy comprising the administration of an antibody that blocks IL-4 signaling (e.g., dupilumab) with an immune checkpoint inhibitor, for example, a PD-(L)1 blocking agent (e.g., pembrolizumab). In one embodiment, the present disclosure provides a method of treating cancer comprising administering to a subject a first dose of about 600 mg of dupilumab followed by one or more successive doses of about 300 mg of dupilumab about every three weeks thereafter, wherein the subject was treated or is undergoing treatment with a PD-1 or PD-L1 blocking agent.

It will be appreciated that antibodies that block PD-(L)1 signaling may including (a) those antibodies that are specific for the PD-1 immune checkpoint protein on the surface of activated T cells and which block the binding of cancer-expressed PD-L1 or PD-L2 (programmed cell death 1 ligand 1 or programmed cell death 1 ligand 2). The binding of PD-L1 or PD-L2 result in the activated T cell from becoming inactive and consequently, ineffective in killing the cancer cell. Thus, the PD-L1 or PD-L2 cancer cell-expressed proteins effectively shut down the body's natural cancer immunity that would otherwise lead to the apoptotic destruction of the cancer cell. In other embodiments, the immune checkpoint inhibitors may also be antibodies that specifically target and bind to the PD-L1 and PD-L2 proteins produced by the cancer cells.

As used herein, the term “immune checkpoint” refers to a regulator of the immune system that affects immune system self-tolerance and helps prevent the immune system from attacking cells indiscriminately. For example, when the immune system is attacking pathogens, immune checkpoint molecules can protect normal tissues from being damaged by the immune response against the pathogens. Some cancer cells have leveraged immune checkpoints to escape from immune system attack by dysregulating immune checkpoint proteins, which include, but are not limited to cytotoxic T-lymphocyte-associated protein 4 (CTLA-4), programmed cell death protein 1 (PD-1), programmed cell death ligand 1 (PD-L1), and programmed cell death ligand 2 (PD-L2). Immune checkpoints are targets for cancer immunotherapy due to their potential for use in multiple types of cancers. Current checkpoint inhibitors approved in the U.S. by the FDA include those that block CTLA4, PD-1, and/or PD-L1.

As used herein, the term “immune checkpoint inhibitor” (“ICI”) or equivalently, “checkpoint inhibitor.” refers to an agent which targets immune checkpoint proteins (e.g., PD-1 or CTLA-4 or their ligands) and inhibits and/or reverses the checkpoint blockade on the immune system caused by the checkpoint protein (e.g., PD-1 or CTLA-4). In other words, the agent blocks the interaction (e.g., between PD-1 and PD-L1) between the checkpoint protein (e.g., PD-1) and the checkpoint blockade inhibitor (e.g., PD-L1), thereby restoring and/or maintaining the T cells in an activated cancer-killing state. In some embodiments, the immune checkpoint inhibitor can bind to CTLA-4, thereby inhibiting the immune checkpoint protein from inhibiting the immune system. In other embodiments, the immune checkpoint inhibitor can bind to PD-1, thereby inhibiting the immune checkpoint protein from inhibiting the immune system. In other embodiments the immune checkpoint inhibitor can bind to PD-L1 (produced by cancer cell), thereby blocking the PD-L1 from binding to PD-1, and consequently blocking the PD-1/PD-L1 interaction and avoiding the effects of PD-L1 on PD-1 on lowering the activity of the immune system (and consequently, more effective against killing cancer cells).

In other words, immune checkpoints are a normal part of the immune system. Their role is to prevent an immune response from being so strong that it destroys healthy cells in the body.

Immune checkpoints engage when proteins on the surface of T cells recognize and bind to partner proteins on other cells, such as some tumor cells. These proteins are called immune checkpoint proteins. When the checkpoint and partner proteins bind together, they send an “off” signal to the T cells. This can prevent the immune system from destroying the cancer.

Immunotherapy drugs called immune checkpoint inhibitors work by blocking checkpoint proteins from binding with their partner proteins. This prevents the “off” signal from being sent, allowing the T cells to kill cancer cells.

Immune system checkpoints operate to modulate (e.g., increase, decrease, start, stop, etc.) immunologic activity (e.g., immune response to an immunologic stimulus). In the case where a disorder (e.g., indication, disease, for example cancer) which exploits immune checkpoints (i.e., blocks them so as to decrease the host immune response to the disorder (e.g., immunologic stimulus)), inhibitors (e.g., checkpoint inhibitors) may be advantageously used to inhibit the effect of the checkpoints, thus decreasing the inhibitory effect of the checkpoint (i.e., increasing or restoring the immunologic response to the disorder). Non-limiting examples of checkpoint inhibitors include, but are not limited to (1) ipilimumab (YERVOY®), an anti-CTLA-4 antibody, (2) tremelimumab, an anti-CTLA-4 antibody, (3) nivolumab (OPDIVO®), an anti-PD-1 antibody, (4) pembrolizumab (KEYTRUDA®), an anti-PD-1 antibody, (5) pidilizumab, and anti-PD-1 antibody, (6) MEDI0680, an anti-PD-1 antibody, (7) atezolizumab, an anti-PD-L1 antibody, (8) avelumab, an anti-PD-L1 antibody, (9) durvalumab, an anti-PD-L1 antibody, and (10) cemiplimab (LIBTAYO®), an anti-PD-1 antibody.

The following exemplary agents for inhibiting immune checkpoints may be used in combination with one or more agents that block IL-4 signaling.

### Anti-CTLA-4 Antibodies

### Ipilimumab

Ipilimumab is an anti-CTLA-4 monoclonal antibody (IgG1 subtype) and immune checkpoint inhibitor approved in 2011 and sold in the U.S. as YERVOY® as a treatment for melanoma. It is undergoing clinical trials for use as a treatment in other cancers. Administration of ipilimumab works to activate the immune system by targeting CTLA-4, a protein receptor expressed on the surface of activated T lymphocytes and which downregulates the T cells' ability to kill cancer cells.

### Tremelimumab

Tremelimumab is an anti-CTLA-4 monoclonal antibody (IgG2 subtype) and immune checkpoint inhibitor under clinical study in the U.S. for treatment of melanoma, mesothelioma, and non-small cell lung cancer. Administration of tremelimumab works to activate the immune system by targeting CTLA-4, a protein receptor expressed on the surface of activated T lymphocytes and which downregulates the T cells' ability to kill cancer cells.

### Anti-PD-1 Antibodies

### Nivolumab

Nivolumab is an anti-PD-1 antibody and immune checkpoint inhibitor approved in the U.S. as OPDIVO® for the treatment of a variety of cancers, including melanoma, lung cancer, mesothelioma, head and neck cancer, colon cancer, and liver cancer, among others. Administration of nivolumab acts to block PD-(L)1 checkpoint inhibitor signaling, thereby activating the immune system to better kill cancer cells. Specifically, PD-1 is a protein on the surface of activated T cells. If another molecule, called programmed cell death 1 ligand 1 or programmed cell death 1 ligand 2 (PD-L1 or PD-L2) binds to PD-1, the initially-activated T cell becomes inactive. This is one way that the body regulates the immune system, to avoid an overreaction. Many cancer cells make PD-L1, which inhibits T cells from attacking the tumor. Nivolumab blocks PD-L1 from binding to PD-1, allowing the T cell to work against the cancer cells.

### Pembrolizumab

Pembrolizumab is an anti-PD-1 antibody and immune checkpoint inhibitor approved in the U.S. in 2014 as KEYTRUDA® for the treatment of a variety of cancers, including melanoma, NSCLC, and mesothelioma, among others. Administration of pembrolizumab acts to block PD-(L)1 checkpoint inhibitor signaling, thereby activating the immune system to better kill cancer cells. Specifically, PD-1 is a protein on the surface of activated T cells. If another molecule, called programmed cell death 1 ligand 1 or programmed cell death 1 ligand 2 (PD-L1 or PD-L2) binds to PD-1, the initially-activated T cell becomes inactive. This is one way that the body regulates the immune system, to avoid an overreaction. Many cancer cells make PD-L1, which inhibits T cells from attacking the tumor. Pembrolizumab blocks PD-L1 from binding to PD-1, allowing the T cell to work against the cancer cells.

### Pidilizumab

Pidilizumab is an anti-PD-1 monoclonal antibody and immune checkpoint inhibitor under clinical study in the U.S. for the treatment of a variety of cancers, including melanoma, NSCLC, and mesothelioma, among others. Administration of pidilizumab acts to block PD-(L)1 checkpoint inhibitor signaling, thereby activating the immune system to better kill cancer cells. Specifically, PD-1 is a protein on the surface of activated T cells. If another molecule, called programmed cell death 1 ligand 1 or programmed cell death 1 ligand 2 (PD-L1 or PD-L2) binds to PD-1, the initially-activated T cell becomes inactive. This is one way that the body regulates the immune system, to avoid an overreaction. Many cancer cells make PD-L1, which inhibits T cells from attacking the tumor. Pidilizumab blocks PD-L1 from binding to PD-1, allowing the T cell to work against the cancer cells.

### Cemiplimab

Cemiplimab is an anti-PD-1 antibody and immune checkpoint inhibitor approved in the U.S. in 2018 as LIBTAYO® for the treatment of basal cell carcinoma, cutaneous squamous cell skin cancer, and non-small cell lung cancer. It is under clinical study for use in a variety of other cancers, including myeloma, advanced cervical cancer, and lung cancer. Administration of cemiplimab acts to block PD-(L)1 checkpoint inhibitor signaling, thereby activating the immune system to better kill cancer cells. Specifically, PD-1 is a protein on the surface of activated T cells. If another molecule, called programmed cell death 1 ligand 1 or programmed cell death 1 ligand 2 (PD-L1 or PD-L2) binds to PD-1, the initially-activated T cell becomes inactive. This is one way that the body regulates the immune system, to avoid an overreaction. Many cancer cells make PD-L1, which inhibits T cells from attacking the tumor. Cemiplimab blocks PD-L1 from binding to

### Anti-PD-L1 Antibodies

### Durvalumab

Durvalumab is a programmed death-ligand 1 (PD-L1) blocking antibody which was approved by the FDA in 2017 for the treatment of unresectable stage 3 non-small cell lung cancer (NSCLC) after concurrent chemoradiation therapy (cCRT). It is a human immunoglobulin G1 kappa (IgG1K) monoclonal antibody that blocks the interaction of programmed cell death ligand 1 (PD-L1) with the PD-1 (CD279). Expression of PD-L1 by cancer cells can be induced by inflammatory signals (e.g., IFN-gamma) and can be expressed on both tumor cells and tumor-associated immune cells in the tumor microenvironment. PD-L1 blocks T-cell function and activation through interaction with PD-1 and CD80. By binding to its receptors, PD-L1 reduces cytotoxic T-cell activity, proliferation, and cytokine production. Blockade of PD-L1/PD-1 and PD-L1/CD80 interactions releases the inhibition of immune responses, without inducing antibody dependent cell-mediated cytotoxicity.

### Atezolizumab

Atezolizumab blocks the interaction of PD-L1 with programmed cell death protein 1 (PD-1) and CD80 receptors (B7-1Rs). It is FDA-approved for treating certain cancers, including urothelial carcinoma, non-small cell lung cancer (NSCLC), triple-negative breast cancer (TNBC), small cell lung cancer (SCLC), and hepatocellular carcinoma (HCC). It is a fully humanized, engineered monoclonal antibody of IgG1 isotype against the protein programmed cell death-ligand 1 (PD-L1) and sold as TECENTRIQ®. PD-L1 can be highly expressed on certain tumors, which is thought to lead to reduced activation of immune cells (cytotoxic T-cells in particular) that might otherwise recognize and attack the cancer. Inhibition of PD-L1 by atezolizumab can remove this inhibitor effect and thereby engender an anti-tumor response. It is one of several ways to block inhibitory signals related to T-cell activation, a more general strategy known as “immune checkpoint inhibition.”

### Avelumab

Avelumab blocks the interaction of PD-L1 with programmed cell death protein 1 (PD-1) and CD80 receptors (B7-1Rs). It is FDA-approved for treating certain cancers, including Merkel cell carcinoma, urothelial carcinoma, and renal cell carcinoma. It is a fully humanized, engineered monoclonal antibody of IgG1 isotype against the protein programmed cell death-ligand 1 (PD-L1) and sold as BAVENCIO®. PD-L1 can be highly expressed on certain tumors, which is thought to lead to reduced activation of immune cells (cytotoxic T-cells in particular) that might otherwise recognize and attack the cancer. Inhibition of PD-L1 by atezolizumab can remove this inhibitor effect and thereby engender an anti-tumor response. It is one of several ways to block inhibitory signals related to T-cell activation, a more general strategy known as “immune checkpoint inhibition.”

As is appreciated by the skilled artisan, antibodies may be active for the intended binding when less than all of the protein comprising the antibody is present, as well as when there are variations (e.g., mutations, substitutions, deletions, insertions, etc.) in the amino acid sequence comprising the protein, or nucleic acid encoding the protein. Accordingly, in some embodiments, the disclosure relates to the use of proteins with less than 100% identity to those expressly disclosed herein. The evaluation of such proteins to ascertain their appropriateness for use in the methods of the present disclosure are well within the skill of the skilled artisan, and evaluated without undue experimentation.

In some embodiments, an anti-PD-(L)1 antibody as used in accordance with the present disclosure comprises a protein or polypeptide comprising an amino acid sequence that is at least 70% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 99.95%, 99.99%, or more) identical to the amino acid sequence of KEYTRUDA® (pembrolizumab). In some embodiments, an anti-PD-1 antibody as used in accordance with the present disclosure comprises a protein or polypeptide comprising an amino acid sequence of KEYTRUDA® (pembrolizumab). In some embodiments, an anti-PD-1 antibody as used in accordance with the present disclosure comprises a protein or polypeptide comprising an amino acid sequence that is at least 70% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 99.95%, 99.99%, or more) identical to the amino acid sequence of OPDIVO® (nivolumab). In some embodiments, an anti-PD-1 antibody as used in accordance with the present disclosure comprises a protein or polypeptide comprising an amino acid sequence of OPDIVO® (nivolumab). In some embodiments, an anti-PD-1 antibody as used in accordance with the present disclosure comprises a protein or polypeptide comprising an amino acid sequence that is at least 70% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 99.95%, 99.99%, or more) identical to the amino acid sequence of LIBTAYO® (cemiplimab). In some embodiments, an anti-PD-L1 antibody as used in accordance with the present disclosure comprises a protein or polypeptide comprising an amino acid sequence of LIBTAYO® (cemiplimab).

In some embodiments, an anti-PD-L1 antibody as used in accordance with the present disclosure comprises a protein or polypeptide comprising an amino acid sequence that is at least 70% (e.g., 70%, 71%, 72%, 73%, 74%, 75%, 76%, 77%, 78%. 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 99.95%, 99.99%, or more) identical to the amino acid sequence of TECENTRIQ® (atezolizumab). In some embodiments, an anti-PD-L1 antibody as used in accordance with the present disclosure comprises a protein or polypeptide comprising an amino acid sequence of TECENTRIQ® (atezolizumab). In some embodiments, an anti-PD-L1 antibody as used in accordance with the present disclosure comprises a protein or polypeptide comprising an amino acid sequence that is at least 70% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 99.95%, 99.99%, or more) identical to the amino acid sequence of BAVENCIO® (avelumab). In some embodiments, an anti-PD-L1 antibody as used in accordance with the present disclosure comprises a protein or polypeptide comprising an amino acid sequence of BAVENCIO® (avelumab). In some embodiments, an anti-PDL-1 antibody as used in accordance with the present disclosure comprises a protein or polypeptide comprising an amino acid sequence that is at least 70% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 99.95%, 99.99%, or more) identical to the amino acid sequence of IMFINZI® (durvalumab). In some embodiments, an anti-IL-4 antibody as used in accordance with the present disclosure comprises a protein or polypeptide comprising an amino acid sequence of IMFINZI® (durvalumab).

### C. Anti-IL-4 Signaling Antibodies

The present inventors have discovered that immunotherapies that block the PD-1/PD-L1 immune checkpoint axis may be made more effective with the co-administration of a specific dosing regimen of an antibody that disrupts IL-4 signaling. The dosing amounts, frequency of administration, and spacing between multiple administrations may all vary. In one embodiment, the dosing regimen comprises administering about 600 mg of dupilumab as a first dose, followed by two subsequent doses of about 300 mg of dupilumab about every three weeks thereafter.

The methods and compositions in this disclosure relate to IL-4, and in particular, IL-4 signaling blockage by administering anti-IL-4 or anti-IL-4R antibodies. The terms “interleukin-4” and “IL-4.” as may be used interchangeably herein, refer to a cytokine that specifically binds to IL-4 receptor (IL-4R). In some embodiments, IL-4 is a mammalian IL-4 cytokine. In some embodiments, IL-4 is a human IL-4 cytokine. Without being bound by theory, IL-4 has many biological roles, including the stimulation of activated B-cell and T-cell proliferation, and the differentiation of B cells into plasma cells. It is a key regulator in humoral and adaptive immunity. IL-4 induces B-cell class switching to IgE, and up-regulates MHC class II production. IL-4 is recognized as playing a pivotal role in shaping the nature of immune responses.

Interleukin-4 plays a critical role in initiating and regulating Th2-type immune responses. In mice, IL-4 is a 14-19 kDa glycoprotein localized on chromosome 11, together with the genes for IL-5 and IL-13. During the innate immune response, evidence suggests that early IL-4-producers include basophils, mast cells, eosinophils, natural killer (NK) T cells, and innate-like skin keratinocytes. T and B lymphocytes orchestrating adaptive immunity, specifically CD4+ Th2 cells, B effector 2 (Be2) B cells, and γ/δ T cells, also secrete IL-4. Apart from regulating the differentiation of Th2 cells, IL-4 also controls immunoglobulin class switching in activated B cells, specifying human B cells to switch to the expression of IgE and IgG4, while in mice, to IgE and IgG1, with the concomitant suppression of IgM, IgG2a, and IgG2b. Moreover, alternatively activated macrophages are activated by IL-4 signaling through the IL-4Rα chain. Importantly, IL-4 inhibits inducible nitric oxide synthase (iNOS) expression thereby inhibiting IFN-γ-induced classically activated macrophages and induction of a type 1 response. As a whole, IL-4 counter-regulates the expression of IFN-γ and increases the expression of MHC II molecules, co-stimulatory molecules CD80 and CD86, and the IL-4 receptor. Reports have also indicated that dendritic cells (DCs) can respond to IL-4 in vivo and in vitro and become alternatively activated, in a manner similar to that described for alternatively activated macrophages, by upregulating multiple alternative activation markers such as mannose receptor and RELM-α. Moreover, although IL-4 has been shown to be the primary inducer of Th2 responses, studies have reported IL-4-independent Th2 differentiation, Th2 cytokine production, IL-4Rα signaling, and STAT6 regulation.

Anti-IL-4 antibodies may be prepared in accordance with the herein methods. Any known IL-4 variant may be used, including the following exemplary IL-4 amino acid sequences, or amino acid sequences having at least 80%, or at least 85%, or at least 90%, or at least 95%, or at least 99% sequence identity with the following sequence:

Human IL-4 (UniProtKB-Q5FC01)
(SEQ ID NO: 1)
MGLTSQLLPP LFFLLACAGN FVHGHKCDIT LQEIIKTLNS
LTEQKNTTEK ETFCRAATVL RQFYSHHEKD TRCLGATAQQ
FHRHKQLIRF LKRLDRNLWG LAGLNSCPVK EANQSTLENF
LERLKTIMRE KYSKCSS
Mouse IL-4 (UniProtKB-P07750)
(SEQ ID NO: 2)
MGLNPQLVVI LLFFLECTRS HIHGCDKNHL REIIGILNEV
TGEGTPCTEM DVPNVLTATK NTTESELVCR ASKVLRIFYL
KHGKTPCLKK NSSVLMELQR LFRAFRCLDS SISCTMNESK
STSLKDFLES LKSIMQMDYS
Rat IL-4 (UniProtKB-P20096)
(SEQ ID NO: 3)
MGLSPHLAVT LFCFLICTGN GIHGCNDSPL REIINTLNQV
TEKGTPCTEM FVPDVLTATR NTTENELICR ASRVLRKFYF
PRDVPPCLKN KSGVLGELRK LCRGVSGLNS LRSCTVNEST
LTTLKDFLES LKSILRGKYL QSCTSMS

In various embodiments, the disclosure provides methods for administering an effective amount of an anti-IL-4 signaling antibody, optionally in combination with an immune checkpoint inhibitor.

In some embodiments, the IL-4 blockade is achieved by blocking the “interleukin-4 receptor,” or equivalently, IL-4R or IL-4Rα, from binding to IL-4 by administering antibodies that bind to the IL-4R. The interleukin-4 receptor is more formally known as the “interleukin-4 receptor subunit alpha,” encoded by the IL4R gene.

IL-4R, a type I transmembrane protein, can bind interleukin-4 and interleukin-13 to regulate IgE antibody production in B cells. Among T cells, the encoded protein also can bind interleukin-4 to promote differentiation of Th2 cells. Allelic variations in this gene have been associated with atopy, a condition that can manifest itself as allergic rhinitis, sinusitis, asthma, or eczema. Two transcript variants encoding different isoforms, a membrane-bound and a soluble form, have been found for this gene. Interactions of IL-4 with TNFα promote structural changes to vascular endothelial cells, thus playing an important role in tissue inflammation.

The binding of IL-4 or IL-13 to the IL-4 receptor on the surface of macrophages results in the alternative activation of those macrophages.

Antibodies that bind to the IL-4R are abundant in the art and well known. Nevertheless, the skill artisan may also prepare antibodies that specifically bind to IL-4R in accordance with this specification based on any IL-4R polypeptide, which may include in a non-limiting manner:

Human IL-4R subunit alpha (UniProtKB-P24394)
(SEQ ID NO: 4)
MGWLCSGLLF PVSCLVLLQV ASSGNMKVLQ EPTCVSDYMS
ISTCEWKMNG PTNCSTELRL LYQLVFLLSE AHTCIPENNG
GAGCVCHLLM DDVVSADNYT LDLWAGQQLL WKGSFKPSEH
VKPRAPGNLT VHTNVSDTLL LTWSNPYPPD NYLYNHLTYA
VNIWSENDPA DFRIYNVTYL EPSLRIAAST LKSGISYRAR
VRAWAQCYNT TWSEWSPSTK WHNSYREPFE QHLLLGVSVS
CIVILAVCLL CYVSITKIKK EWWDQIPNPA RSRLVAIIIQ
DAQGSQWEKR SRGQEPAKCP HWKNCLTKLL PCFLEHNMKR
DEDPHKAAKE MPFQGSGKSA WCPVEISKTV LWPESISVVR
CVELFEAPVE CEEEEEVEEE KGSFCASPES SRDDFQEGRE
GIVARLTESL FLDLLGEENG GFCQQDMGES CLLPPSGSTS
AHMPWDEFPS AGPKEAPPWG KEQPLHLEPS PPASPTQSPD
NLTCTETPLV IAGNPAYRSF SNSLSQSPCP RELGPDPLLA
RHLEEVEPEM PCVPQLSEPT TVPQPEPETW EQILRRNVLQ
HGAAAAPVSA PTSGYQEFVH AVEQGGTQAS AVVGLGPPGE
AGYKAFSSLL ASSAVSPEKC GFGASSGEEG YKPFQDLIPG
CPGDPAPVPV PLFTFGLDRE PPRSPQSSHL PSSSPEHLGL
EPGEKVEDMP KPPLPQEQAT DPLVDSLGSG IVYSALTCHL
CGHLKQCHGQ EDGGQTPVMA SPCCGCCCGD RSSPPTTPLR
APDPSPGGVP LEASLCPASL APSGISEKSK SSSSFHPAPG
NAQSSSQTPK IVNFVSVGPT YMRVS.

The term “anti-IL-4 signaling antibody,” or equivalently, “antibodies that block IL-4 signaling,” as may be used herein, refers to antibodies that directly or indirectly block or reduce IL-4 signaling.

Anti-IL-4 signaling antibodies embraces both anti-IL-4 antibodies and anti-IL-4R antibodies since either IL-4 and IL-4R can be targeted to block IL-4 signaling.

In certain embodiments, an anti-IL-4 signaling antibody can be an antibody that specifically binds to IL-4, thereby blocking or reducing the ability of IL-4 from binding the IL-4 receptor. In certain other embodiments, an anti-IL-4 signaling antibody can be an antibody that specifically binds to the IL-4 receptor, thereby blocking or reducing the ability of IL-4 from binding to the IL-4 receptor. Both classes of antibodies are well-known in the art and can obtain from commercial sources, the literature and/or public repositories, expressed from known nucleotide sequences, or even obtained by traditional methods of antibody preparation based on the antigen (e.g., IL-4 or IL-4R).

These antibodies can be obtained, for example, from many commercial suppliers, including, but not limited to INVITROGEN, PROTEINTECH, and ENZO LIFE SCIENCES.

In still other embodiments, anti-IL-4 antibodies and anti-IL-4R antibodies can be prepared by methods well-known in art and discussed below.

The following U.S. patents disclose numerous anti-IL-4 antibodies. All of the antibodies, and related teachings are incorporated herein by reference in their entireties: U.S. Pat. Nos. 10,759,871, 973,728, 9,732,162, 8,735,095, 8,388,965, 8,338,135, 8,337,839, 8,092,802, 8,075,887, 7,794,717, 7,740,843, 7,608,693, 7,605,237, and 5,985,280.

The following U.S. patents disclose numerous anti-IL-4R antibodies. All of the antibodies, and related teachings are incorporated herein by reference in their entireties: U.S. Pat. Nos. 10,435,473, 9,238,692, 8,945,559.

Other strategies for targeting IL4 signaling pathway can be found in Bankaitis et al., “Targeting IL4/IL4R for the treatment of epithelial cancer metastasis,” Clin Exp Metastasis, 2015, 32(8): 847-856.

### Dupilumab

In one embodiment, the anti-IL-4 receptor antibody can be dupilumab, which is marketed under the name DUPIXENT®. Dupilumab binds to the alpha subunit of the interleukin-4 receptor (IL-4Rα), making it a receptor antagonist. Through blockade of IL-4Rα, dupilumab modulates signaling of the IL-4 pathway.

Without being bound by theory, the Th2 cytokines interleukin 4 (IL-4) and IL-13 and the heterodimeric IL-4 receptor (IL-4R) complexes that they interact with play a key role in the pathogenesis of allergic disorders. Dupilumab is a humanized IgG4 monoclonal antibody that targets the IL-4 receptor alpha chain (IL-4Rα), common to both IL-4R complexes: type 1 (IL-4Rα/γc; IL-4 specific) and type 2 (IL-4Rα/IL-13Rα1; IL-4 and IL-13 specific) (see FIG. 3).

In some embodiments, an anti-IL-4R antibody as used in accordance with the present disclosure comprises a protein or polypeptide comprising an amino acid sequence that is at least 70% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 99.95%, 99.99%, or more) identical to the amino acid sequence of DUPIXENT® (dupilumab). In some embodiments, an anti-IL-4R antibody as used in accordance with the present disclosure comprises a protein or polypeptide comprising an amino acid sequence of DUPIXENT® (dupilumab).

As is appreciated by the skilled artisan, antibodies may be active for the intended binding when less that all of the protein comprising the antibody is present, as well as when there are variations (e.g., mutations, substitutions, deletions, insertions, etc.) in the amino acid sequence comprising the protein, or nucleic acid encoding the protein. Accordingly, in some embodiments, the disclosure relates to the use of proteins with less than 100% identity to those expressly disclosed herein. The evaluation of such proteins to ascertain their appropriateness for use in the methods of the present disclosure are well within the skill of the skilled artisan, and evaluated without undue experimentation.

### D. Antibody Modifications

The antibodies (e.g., anti-IL-4 signaling antibodies or immune checkpoint therapies) described herein may comprise various modifications, or may be modified in various ways well known to the skilled artisan. For example, antibodies or antigen binding fragments of the disclosure may be modified with a detectable label, including, but not limited to, an enzyme, prosthetic group, fluorescent material (e.g., green fluorescent protein), luminescent material, bioluminescent material, radioactive material, positron emitting metal, nonradioactive paramagnetic metal ion, and affinity label for detection. The detectable substance may be coupled or conjugated either directly to the polypeptides of the disclosure or indirectly, through an intermediate (such as, for example, a linker) using suitable techniques. Non-limiting examples of suitable enzymes include horseradish peroxidase, alkaline phosphatase, ß-galactosidase, glucose oxidase, or acetylcholinesterase; non-limiting examples of suitable prosthetic group complexes include streptavidin/biotin and avidin/biotin; non-limiting examples of suitable fluorescent materials include biotin, umbelliferone, fluorescein, fluorescein isothiocyanate, rhodamine, dichlorotriazinylamine fluorescein, dansyl chloride, or phycoerythrin; an example of a luminescent material includes luminol; non-limiting examples of bioluminescent materials include luciferase, luciferin, and acquorin; and examples of suitable radioactive material include a radioactive metal ion, e.g., alpha-emitters or other radioisotopes such as, for example, iodine (1311, 1251, 1231, 1211), carbon (14C), sulfur (35S), tritium (3H), indium (115mIn, 113mIn, 112 In, 111 In), and technetium (99Tc, 99mTc), thallium (201Ti), gallium (68Ga, 67Ga), palladium (103Pd), molybdenum (99Mo), xenon (133Xe), fluorine (18F), 153Sm, Lu, 159Gd, 149Pm, 140La, 175Yb, 166Ho, 90Y, 47Sc, 86R, 188Re, 142Pr, 105Rh, 97Ru, 68Ge, 57Co, 65Zn, 85Sr, 32P, 153Gd, 169Yb, 51Cr, 54Mn, 75Se, and tin (113Sn, 117Sn). The detectable substance may be coupled or conjugated either directly to the agents of the disclosure (e.g., IL-4 antibodies) or indirectly, through an intermediate (such as, for example, a linker) using suitable techniques. Antibodies of the disclosure conjugated to a detectable substance may be used for diagnostic assays as described herein.

In some embodiments, any of the antibodies for use in the methods of the instant disclosure (e.g., anti-IL4 antibodies, anti-PD-1 antibodies, anti-PD-L1 antibodies, combinations thereof) may comprise a polyhistidine tag. In some embodiments, the polyhistidine tag comprises two, three, four, five six, seven, eight, nine, ten, or more consecutive histidine residues. In some embodiments, the polyhistidine tag is comprised at the N-terminus of any of the antibodies provided herein. In some embodiments, the polyhistidine tag is comprised at the C-terminus of any of the antibodies provided herein. In some embodiments, the polyhistidine tag is comprised within any of the antibodies provided herein. In some embodiments, the polyhistidine tag is fused directly to any of the antibodies provided herein. In some embodiments, the polyhistidine tag is fused to any of the antibodies provided herein via a linker. While polyhistidine is a common and useful purification tag, it is well known in the art that other expressible peptide sequences can act as tags for both purification and localization of the antibody in later pre-clinical studies. In many instances, use of protein A affinity is used to purify antibodies.

### E. Antibody Production

In various embodiments, the herein disclosed methods comprise the use of FDA-approved and commercially-available antibody therapies. For instance, in one embodiment, the disclosure provides a method of treating cancer comprising administering to a subject a first dose of about 600 mg of dupilumab followed by one or more successive doses of about 300 mg of dupilumab about every three weeks thereafter, wherein the subject was treated or is undergoing treatment with a PD-1 or PD-L1 blocking agent. In certain embodiments, the dupilumab may be obtained as DUPIXENT® and the PD-1 or PD-L1 blocking agent may be obtained as pembrolizumab (KEYTRUDA®).

In other embodiments, the antibodies used herein may be produced by known methods.

Numerous methods may be used for obtaining antibodies, or antigen binding fragments thereof, as in the instant disclosure. For example, antibodies can be produced using recombinant DNA methods. Monoclonal antibodies may also be produced by generation of hybridomas (see e.g., Kohler and Milstein (1975) Nature, 256:495-499) in accordance with known methods. Hybridomas formed in this manner are then screened using standard methods, such as enzyme-linked immunosorbent assay (ELISA) and surface plasmon resonance (e.g., OCTET or BIACORE) analysis, to identify one or more hybridomas that produce an antibody that specifically binds to a specified antigen (e.g., IL-4, or fragment thereof). Any form of the specified antigen may be used as the immunogen, e.g., recombinant antigen, naturally occurring forms, any variants or fragments thereof, as well as antigenic peptide thereof (e.g., any of the epitopes described herein as a linear epitope or within a scaffold as a conformational epitope). One exemplary method of making antibodies includes screening protein expression libraries that express antibodies or fragments thereof (e.g., scFv), e.g., phage or ribosome display libraries. Phage display is described, for example, in Ladner et al., U.S. Pat. No. 5,223,409; Smith (1985) Science 228:1315-1317; Clackson et al., (1991) Nature, 352:624-628; Marks et al., (1991) J. Mol. Biol., 222:581-597WO92/18619; WO 91/17271; WO 92/20791; WO 92/15679; WO 93/01288; WO 92/01047; WO 92/09690; and WO 90/02809.

In addition to the use of display libraries, the specified antigen (e.g., IL-4, fragments thereof) can be used to immunize a subject (e.g., non-human animal, e.g., a rodent, e.g., a mouse, hamster, or rat). In one embodiment, a non-human animal is a mouse.

In another embodiment, a monoclonal antibody is obtained from a non-human animal, and then modified (e.g., made chimeric) using suitable recombinant DNA techniques. A variety of approaches for making chimeric antibodies have been described. See e.g., Morrison et al., Proc. Natl. Acad. Sci. U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B.

For additional antibody production techniques, see Antibodies: A Laboratory Manual, eds. Harlow et al., Cold Spring Harbor Laboratory, 1988. The present disclosure is not necessarily limited to any particular source, method of production, or other special characteristics of an antibody.

Some aspects of the present disclosure relate to host cells transformed with a polynucleotide or vector. Host cells may be a prokaryotic or eukaryotic cell. The polynucleotide or vector which is present in the host cell may either be integrated into the genome of the host cell or it may be maintained extrachromosomally. The host cell can be any prokaryotic or eukaryotic cell, such as a bacterial, insect, fungal, plant, animal or human cell. In some embodiments, fungal cells are, for example, those of the genus Saccharomyces, in particular those of the species S. cerevisiae. The term “prokaryotic” includes all bacteria which can be transformed or transfected with a DNA or RNA molecules for the expression of an antibody or the corresponding immunoglobulin chains. Prokaryotic hosts may include gram negative as well as gram positive bacteria such as, for example, E. coli, S. typhimurium, Serratia marcescens, and Bacillus subtilis. The term “eukaryotic” includes yeast, higher plants, insects and vertebrate cells, e.g., mammalian cells, such as NSO and CHO cells. Depending upon the host employed in a recombinant production procedure, the antibodies or immunoglobulin chains encoded by the polynucleotide may be glycosylated or may be non-glycosylated. Antibodies or the corresponding immunoglobulin chains may also include an initial methionine amino acid residue.

In some embodiments, once a vector has been incorporated into an appropriate host, the host may be maintained under conditions suitable for high level expression of the nucleotide sequences, and, as desired, the collection and purification of the immunoglobulin light chains, heavy chains, light/heavy chain dimers, or intact antibodies, antigen binding fragments or other immunoglobulin forms may follow; see, Beychok, Cells of Immunoglobulin Synthesis, Academic Press, N.Y., (1979). Thus, polynucleotides or vectors are introduced into the cells which in turn produce the antibody or antigen binding fragments. Furthermore, transgenic animals, preferably mammals, comprising the aforementioned host cells may be used for the large-scale production of the antibody or antibody fragments.

The transformed host cells can be grown in fermenters and cultured using any suitable techniques to achieve optimal cell growth. Once expressed, the whole antibodies, their dimers, individual light and heavy chains, other immunoglobulin forms, or antigen binding fragments, can be purified according to standard procedures of the art, including ammonium sulfate precipitation, affinity columns, column chromatography, gel electrophoresis and the like; see, Scopes, “Protein Purification”, Springer Verlag, N.Y. (1982). The antibody or antigen binding fragments can then be isolated from the growth medium, cellular lysates, or cellular membrane fractions. The isolation and purification of the, e.g., microbially expressed antibodies or antigen binding fragments may be by any conventional means such as, for example, preparative chromatographic separations and immunological separations such as those involving the use of monoclonal or polyclonal antibodies directed, e.g., against the constant region of the antibody.

Aspects of the disclosure relate to a hybridoma, which provides an indefinitely prolonged source of monoclonal antibodies. As an alternative to obtaining immunoglobulins directly from the culture of hybridomas, immortalized hybridoma cells can be used as a source of rearranged heavy chain and light chain loci for subsequent expression and/or genetic manipulation. Rearranged antibody genes can be reverse transcribed from appropriate mRNAs to produce cDNA. In some embodiments, heavy chain constant region can be exchanged for that of a different isotype or eliminated altogether. The variable regions can be linked to encode single chain Fv regions. Multiple Fv regions can be linked to confer binding ability to more than one target or chimeric heavy and light chain combinations can be employed. Any appropriate method may be used for cloning of antibody variable regions and generation of recombinant antibodies.

In some embodiments, an appropriate nucleic acid that encodes variable regions of a heavy chain and/or light chain is obtained and inserted into an expression vectors which can be transfected into standard recombinant host cells. A variety of such host cells may be used. In some embodiments, mammalian host cells may be advantageous for efficient processing and production. Typical mammalian cell lines useful for this purpose include CHO cells, 293 cells, or NSO cells. The production of the antibody or antigen binding fragment may be undertaken by culturing a modified recombinant host under culture conditions appropriate for the growth of the host cells and the expression of the coding sequences. The antibodies or antigen binding fragments may be recovered by isolating them from the culture. The expression systems may be designed to include signal peptides so that the resulting antibodies are secreted into the medium; however, intracellular production is also possible.

The disclosure also includes a polynucleotide encoding at least a variable region of an immunoglobulin chain of any of the antibodies described herein. In some embodiments, the variable region encoded by the polynucleotide comprises at least one complementarity determining region (CDR) of the VH and/or VL of the variable region of the antibody produced by any one of the above described hybridomas.

Polynucleotides encoding antibody or antigen binding fragments may be, e.g., DNA, cDNA, RNA or synthetically produced DNA or RNA or a recombinantly produced chimeric nucleic acid molecule comprising any of those polynucleotides either alone or in combination. In some embodiments, a polynucleotide is part of a vector. Such vectors may comprise further genes such as marker genes which allow for the selection of the vector in a suitable host cell and under suitable conditions.

In some embodiments, a polynucleotide is operatively linked to expression control sequences allowing expression in prokaryotic or eukaryotic cells. Expression of the polynucleotide comprises transcription of the polynucleotide into a translatable messenger RNA (mRNA). Regulatory elements ensuring expression in eukaryotic cells, preferably mammalian cells, are well known to those skilled in the art. They may include regulatory sequences that facilitate initiation of transcription and optionally polyadenylation (poly-A) signals that facilitate termination of transcription and stabilization of the transcript. Additional regulatory elements may include transcriptional as well as translational enhancers, and/or naturally associated or heterologous promoter regions. Possible regulatory elements permitting expression in prokaryotic host cells include, e.g., the PL, Lac, Trp or Tac promoter in E. coli, and examples of regulatory elements permitting expression in eukaryotic host cells are the AOX1 or GAL1 promoter in yeast or the CMV-promoter, SV40-promoter, RSV-promoter (Rous sarcoma virus), CMV-enhancer, SV40-enhancer, or a globin intron in mammalian and other animal cells.

Beside elements which are responsible for the initiation of transcription such regulatory elements may also include transcription termination signals, such as the SV40-poly-A site or the tk-poly-A site, downstream of the polynucleotide. Furthermore, depending on the expression system employed, leader sequences capable of directing the polypeptide to a cellular compartment or secreting it into the medium may be added to the coding sequence of the polynucleotide and have been described previously. The leader sequence(s) is (are) assembled in appropriate phase with translation, initiation and termination sequences, and preferably, a leader sequence capable of directing secretion of translated protein, or a portion thereof, into, for example, the extracellular medium. Optionally, a heterologous polynucleotide sequence can be used that encode a fusion protein including an N-terminal (amine-end) or C-terminal (carboxyl-end) identification peptide imparting desired characteristics, e.g., stabilization or simplified purification of expressed recombinant product.

In some embodiments, polynucleotides encoding at least the variable domain of the light chain and/or heavy chain may encode the variable domains of both immunoglobulin chains or only one. Likewise, polynucleotides may be under the control of the same promoter or may be separately controlled for expression. Furthermore, some aspects of the disclosure relate to vectors, particularly plasmids, cosmids, viruses, and bacteriophages used conventionally in genetic engineering that comprise a polynucleotide encoding a variable domain of an immunoglobulin chain of an antibody or antigen binding fragment; optionally in combination with a polynucleotide that encodes the variable domain of the other immunoglobulin chain of the antibody.

In some embodiments, expression control sequences are provided as eukaryotic promoter systems in vectors capable of transforming or transfecting eukaryotic host cells, but control sequences for prokaryotic hosts may also be used. Expression vectors derived from viruses such as retroviruses, vaccinia virus, adeno-associated virus, herpes viruses, or bovine papilloma virus, may be used for delivery of the polynucleotides or vector into targeted cell population (e.g., to engineer a cell to express an antibody or antigen binding fragment). A variety of appropriate methods can be used to construct recombinant viral vectors. In some embodiments, polynucleotides and vectors can be reconstituted into liposomes for delivery to target cells. The vectors containing the polynucleotides (e.g., the heavy and/or light variable domain(s) of the immunoglobulin chains encoding sequences and expression control sequences) can be transferred into the host cell by suitable methods, which vary depending on the type of cellular host.

Accordingly, it is envisioned that in addition to those compositions expressly disclosed herein, using the instant disclosure, the knowledge and techniques know to the skilled artisan, one of ordinary skill in the art may use the methods and teachings herein to evaluate and derive additional compositions (e.g., proteins, antibodies (e.g., anti-IL-4 antibodies)) which are appropriate for use in connection with the methods disclosed herein. Furthermore, it is envisioned that in addition to those compositions expressly disclosed herein, using the instant disclosure, the knowledge and techniques know to the skilled artisan, one of ordinary skill in the art may use the methods and teachings herein to evaluate and derive additional compositions (e.g., nucleic acids, polynucleotides, vectors, plasmids, hybridomas, etc.) which may be used to create the proteins (e.g., anti-IL-4 antibodies) of the present disclosure. Such compositions are envisioned as within the scope of the embodiments herein. In some embodiments, the disclosure relates to the generation of anti-IL-4 antibodies useful for the methods described herein.

### F. Pharmaceutical Compositions

In various embodiments, the disclosure provides methods for augmenting an immune checkpoint therapy in a subject having cancer, comprising administering to the subject a first dose of about 600 mg of dupilumab followed by one or more successive doses of about 300 mg of dupilumab about every three weeks thereafter, wherein the immune checkpoint therapy comprises a PD-(L)1 blocking agent. Thus, the disclosure provides combination therapies involving (a) antibodies that block or inhibit the PD-1/PD-L1 axis (e.g., pembrolizumab) together with (b) antibodies that block or inhibit IL-4 signaling (e.g. dupilumab) in one or more effective dosage regimens described herein.

The methods disclosed herein may involve one or more pharmaceutical compositions. In some embodiments, the antibodies that block or inhibit the PD-1/PD-L1 axis (e.g., pembrolizumab) are formulated as a separate pharmaceutical composition from the pharmaceutical composition comprising the antibodies that block or inhibit IL-4 signaling (e.g, dupilumab) in one or more effective dosage regimens. In some other embodiments, the antibodies that block or inhibit the PD-1/PD-L1 axis (e.g., pembrolizumab) are formulated as a single pharmaceutical composition that also includes the antibodies that block or inhibit IL-4 signaling (e.g., dupilumab) in one or more effective dosage regimens.

In addition, the pharmaceutical compositions may comprise a single dose of antibodies, or multiple doses of antibodies. The doses may be all the same or may vary. For instance, the disclosure in certain embodiments may comprise a pharmaceutical composition comprising a single dose of the antibodies that block or inhibit IL-4 signaling (e.g, dupilumab). In other embodiments, the disclosure provides a pharmaceutical composition comprising at least two doses, at least three doses, at least four doses, at least five doses, at least six doses, at least seven doses, at least eight doses, at least nine doses, at least ten doses, between 2-5 doses, between 3-10 doses, between 4-20 doses, between 5-30 doses, between 6-40 doses, between 7-50 doses, between 8-60 doses, between 9-70 doses, or between 10-80 doses of the antibodies that block or inhibit IL-4 signaling (e.g. dupilumab).

In the case of either the antibodies that block or inhibit IL-4 signaling (e.g. dupilumab) or the antibodies that block or inhibit the PD-1/PD-L1 axis (e.g., pembrolizumab), administration may be pursuant to SOC (“standard of care”) as understood in the medical field by an ordinarily skilled prescribing medical doctor.

Each dose of the antibodies that block or inhibit IL-4 signaling (e.g. dupilumab) may be 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, or 1000 mg, or any combinations of such doses if the composition includes multiple doses. In one embodiment, the dose is 300 mg. In another embodiment, the dose is 600 mg. In some embodiments, the doses include one or more 300 mg doses and a 600 mg dose.

In other embodiments, each dose of the antibodies that block or inhibit IL-4 signaling (e.g, dupilumab) may be about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, or about 1000 mg, or any combinations of such doses if the composition includes multiple doses. In one embodiment, the dose is 300 mg or about 300 mg. In another embodiment, the dose is 600 mg or about 600 mg. In some embodiments, the doses include one or more about 300 mg doses and an about 600 mg dose.

In other embodiments, each dose of the antibodies that block or inhibit IL-4 signaling (e.g, dupilumab) may be at least about 10 mg, at least about 20 mg, about 30 mg, at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, at least about 200 mg, at least about 210 mg, at least about 220 mg, at least about 230 mg, at least about 240 mg, at least about 250 mg, at least about 260 mg, at least about 270 mg, at least about 280 mg, at least about 290 mg, at least about 300 mg, at least about 310 mg, at least about 320 mg, at least about 330 mg, at least about 340 mg, at least about 350 mg, at least about 360 mg, at least about 370 mg, at least about 380 mg, at least about 390 mg, at least about 400 mg, at least about 410 mg, at least about 420 mg, at least about 430 mg, at least about 440 mg, at least about 450 mg, at least about 460 mg, at least about 470 mg, at least about 480 mg, at least about 490 mg, at least about 500 mg, at least about 510 mg, at least about 520 mg, at least about 530 mg, at least about 540 mg, at least about 550 mg, at least about 560 mg, at least about 570 mg, at least about 580 mg, at least about 590 mg, at least about 600 mg, at least about 610 mg, at least about 620 mg, at least about 630 mg, at least about 640 mg, at least about 650 mg, at least about 660 mg, at least about 670 mg, at least about 680 mg, at least about 690 mg, at least about 700 mg, at least about 710 mg, at least about 720 mg, at least about 730 mg, at least about 740 mg, at least about 750 mg, at least about 760 mg, at least about 770 mg, at least about 780 mg, at least about 790 mg, at least about 800 mg, at least about 810 mg, at least about 820 mg, at least about 830 mg, at least about 840 mg, at least about 850 mg, at least about 860 mg, at least about 870 mg, at least about 880 mg, at least about 890 mg, at least about 900 mg, at least about 910 mg, at least about 920 mg, at least about 930 mg, at least about 940 mg, at least about 950 mg, at least about 960 mg, at least about 970 mg, at least about 980 mg, at least about 990 mg, or at least about 1000 mg but no more than 1001 mg, or any combinations of such doses if the composition includes multiple doses. In one embodiment, the dose is 300 mg or at least about 300 mg. In another embodiment, the dose is 600 mg or about 600 mg. In some embodiments, the doses include one or more about 300 mg doses and an at least about 600 mg dose.

Each dose of the antibodies that block or inhibit the PD-1/PD-L1 axis (e.g., pembrolizumab) may be 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, or 1000 mg, or any combinations of such doses if the composition includes multiple doses. In one embodiment, the dose is 300 mg. In another embodiment, the dose is 600 mg. In some embodiments, the doses include one or more 300 mg doses and a 600 mg dose.

In other embodiments, each dose of the antibodies that block or inhibit the PD-1/PD-L1 axis (e.g., pembrolizumab) may be about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, or about 1000 mg, or any combinations of such doses if the composition includes multiple doses. In one embodiment, the dose is 300 mg or about 300 mg. In another embodiment, the dose is 600 mg or about 600 mg. In some embodiments, the doses include one or more about 300 mg doses and an about 600 mg dose.

In other embodiments, each dose of the antibodies that block or inhibit the PD-1/PD-L1 axis (e.g., pembrolizumab) may be at least about 10 mg, at least about 20 mg, about 30 mg, at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, at least about 200 mg, at least about 210 mg, at least about 220 mg, at least about 230 mg, at least about 240 mg, at least about 250 mg, at least about 260 mg, at least about 270 mg, at least about 280 mg, at least about 290 mg, at least about 300 mg, at least about 310 mg, at least about 320 mg, at least about 330 mg, at least about 340 mg, at least about 350 mg, at least about 360 mg, at least about 370 mg, at least about 380 mg, at least about 390 mg, at least about 400 mg, at least about 410 mg, at least about 420 mg, at least about 430 mg, at least about 440 mg, at least about 450 mg, at least about 460 mg, at least about 470 mg, at least about 480 mg, at least about 490 mg, at least about 500 mg, at least about 510 mg, at least about 520 mg, at least about 530 mg, at least about 540 mg, at least about 550 mg, at least about 560 mg, at least about 570 mg, at least about 580 mg, at least about 590 mg, at least about 600 mg, at least about 610 mg, at least about 620 mg, at least about 630 mg, at least about 640 mg, at least about 650 mg, at least about 660 mg, at least about 670 mg, at least about 680 mg, at least about 690 mg, at least about 700 mg, at least about 710 mg, at least about 720 mg, at least about 730 mg, at least about 740 mg, at least about 750 mg, at least about 760 mg, at least about 770 mg, at least about 780 mg, at least about 790 mg, at least about 800 mg, at least about 810 mg, at least about 820 mg, at least about 830 mg, at least about 840 mg, at least about 850 mg, at least about 860 mg, at least about 870 mg, at least about 880 mg, at least about 890 mg, at least about 900 mg, at least about 910 mg, at least about 920 mg, at least about 930 mg, at least about 940 mg, at least about 950 mg, at least about 960 mg, at least about 970 mg, at least about 980 mg, at least about 990 mg, or at least about 1000 mg but no more than 1001 mg, or any combinations of such doses if the composition includes multiple doses. In one embodiment, the dose is 300 mg or at least about 300 mg. In another embodiment, the dose is 600 mg or about 600 mg. In some embodiments, the doses include one or more about 300 mg doses and an at least about 600 mg dose.

In a particular embodiment, the present inventors have discovered that immunotherapies that block the PD-1/PD-L1 immune checkpoint axis may be made more effective with the co-administration of a specific dosing regimen of an antibody that disrupts IL-4 signaling, wherein the dosing regimen comprises about 600 mg of dupilumab as a first dose, followed by two subsequent doses of about 300 mg of dupilumab about every three weeks thereafter, in combination with ongoing, contemporaneous, or prior standard of care treatment with a immune checkpoint inhibitor, e.g., pembrolizumab. Other dosing regimens are within the scope of the disclosure and are described herein.

For example, the dosing regimen may comprise (a) an ongoing and/or previous treatment with an immune checkpoint inhibitor (e.g., pembrolizumab) and (b) co-administration with a first dose of about 100-120 mg, about 110-130 mg, about 120-140 mg, about 130-150 mg, about 140-160 mg, about 150-170 mg, about 160-180 mg, about 170-190 mg, about 180-200 mg, about 190-210 mg, about 200-220 mg, about 210-230 mg, about 220-240 mg, about 230-250 mg, about 240-260 mg, about 250-270 mg, about 260-280 mg, about 270-290 mg, about 280-300 mg, about 290-310 mg, about 300-320 mg, about 310-320 mg, about 320-340 mg, about 330-350 mg, about 340-360 mg, about 350-370 mg, about 360-380 mg, about 370-390 mg, about 380-400 mg, about 390-410 mg, about 400-420 mg, about 410-430 mg, about 420-440 mg, about 430-450 mg, about 440-460 mg, about 450-470 mg, about 460-480 mg, about 470-490 mg, about 480-500 mg, about 490-510 mg, about 500-520 mg, about 510-530 mg, about 520-540 mg, about 530-550 mg, about 540-560 mg, about 550-570 mg, about 560-580 mg, about 570-590 mg, about 580-600 mg, about 590-610 mg, or about 600-620 mg, followed by at least one subsequent dose of about 100-120 mg, about 110-130 mg, about 120-140 mg, about 130-150 mg, about 140-160 mg, about 150-170 mg, about 160-180 mg, about 170-190 mg, about 180-200 mg, about 190-210 mg, about 200-220 mg, about 210-230 mg, about 220-240 mg, about 230-250 mg, about 240-260 mg, about 250-270 mg, about 260-280 mg, about 270-290 mg, about 280-300 mg, about 290-310 mg, about 300-320 mg, about 310-320 mg, about 320-340 mg, about 330-350 mg, about 340-360 mg, about 350-370 mg, about 360-380 mg, about 370-390 mg, about 380-400 mg, about 390-410 mg, about 400-420 mg, about 410-430 mg, about 420-440 mg, about 430-450 mg, about 440-460 mg, about 450-470 mg, about 460-480 mg, about 470-490 mg, about 480-500 mg, about 490-510 mg, about 500-520 mg, about 510-530 mg, about 520-540 mg, about 530-550 mg, about 540-560 mg, about 550-570 mg, about 560-580 mg, about 570-590 mg, about 580-600 mg, about 590-610 mg, or about 600-620 mg of dupilumab after the first dose of dupilumab.

In other embodiment, additional one, two, three, four, five, six, seven, eight, nine, or ten of more doses of dupilumab may be administered at about 100-120 mg, about 110-130 mg, about 120-140 mg, about 130-150 mg, about 140-160 mg, about 150-170 mg, about 160-180 mg, about 170-190 mg, about 180-200 mg, about 190-210 mg, about 200-220 mg, about 210-230 mg, about 220-240 mg, about 230-250 mg, about 240-260 mg, about 250-270 mg, about 260-280 mg, about 270-290 mg, about 280-300 mg, about 290-310 mg, about 300-320 mg, about 310-320 mg, about 320-340 mg, about 330-350 mg, about 340-360 mg, about 350-370 mg, about 360-380 mg, about 370-390 mg, about 380-400 mg, about 390-410 mg, about 400-420 mg, about 410-430 mg, about 420-440 mg, about 430-450 mg, about 440-460 mg, about 450-470 mg, about 460-480 mg, about 470-490 mg, about 480-500 mg, about 490-510 mg, about 500-520 mg, about 510-530 mg, about 520-540 mg, about 530-550 mg, about 540-560 mg, about 550-570 mg, about 560-580 mg, about 570-590 mg, about 580-600 mg, about 590-610 mg, or about 600-620 mg.

If multiple doses of dupilumab are administered, each of the doses may be the same, or may be different.

In certain embodiments, the spacing between any two doses of dupilumab may be about 1 day, about 2 days, about 3 days, about 4 days, about 5 days, about 6 days, about 7 days, about up to two weeks, about up to three weeks, about up to four weeks, about up to five weeks, about up to six weeks, about up to seven weeks, about up to eight weeks, about up to nine weeks, or about up to ten weeks. In one embodiment, the space between any two doses of dupilumab is about three weeks. In another embodiment, the space between any two doses of dupilumab is exactly three weeks. In still another embodiment, the space between any two doses of dupilumab is exactly 1 day, 2 days, 3 days, 4 days, 5 days, 6 days, 7 days, up to two weeks, up to three weeks, up to four weeks, up to five weeks, up to six weeks, up to seven weeks, up to eight weeks, up to nine weeks, or up to ten weeks.

In other embodiments, the spacing between any multiple doses of dupilumab may be the same or may be different.

In still other embodiments, the disclosure provides a pharmaceutical composition comprising at least two doses, at least three doses, at least four doses, at least five doses, at least six doses, at least seven doses, at least eight doses, at least nine doses, at least ten doses, between 2-5 doses, between 3-10 doses, between 4-20 doses, between 5-30 doses, between 6-40 doses, between 7-50 doses, between 8-60 doses, between 9-70 doses, or between 10-80 doses of the antibodies that block or inhibit IL-4 signaling (e.g, dupilumab).

One or more of the peptides (e.g., proteins, polypeptides, antibodies (e.g., anti-IL-4 antibodies, anti-PD-1 antibodies, anti-PDL-1 antibodies)) can be mixed with a pharmaceutically acceptable carrier (excipient), including buffer, to form a pharmaceutical composition for use accordance with the methods of the present disclosure (e.g., in alleviating a disease or disorder). “Acceptable” means that the carrier must be compatible with the active ingredient of the composition (and preferably, capable of stabilizing the active ingredient) and not deleterious to the subject to be treated, or wherein the deleterious or negative effects are balanced by a medical professional and deemed, using reasonable and sound medical judgment, to be outweighed by the beneficial effects of the composition. Examples of pharmaceutically acceptable excipients (carriers), including buffers, would be apparent to the skilled artisan and have been described previously. See, e.g., Remington: The Science and Practice of Pharmacy 20th Ed. (2000) Lippincott Williams and Wilkins, Ed. K. E. Hoover. In one example, a pharmaceutical composition described herein contains more at least one anti-IL-4 antibody (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more). In some embodiments, a pharmaceutical composition may comprise at least one anti-IL-4 antibody and at least one anti-PD-1 antibody (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more) and/or at least one anti-PDL-1 antibody (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more).

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Pharmaceutically acceptable excipients are further described herein.

In some examples, the pharmaceutical composition described herein comprises liposomes containing the antibodies for use in the methods of the instant disclosure (e.g., anti-IL4 antibodies, anti-PD-1 antibodies, anti-PDL-1 antibodies, combinations thereof), which can be prepared by any suitable method, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The antibodies for use in the methods of the instant disclosure (e.g., anti-IL4 antibodies, anti-PD-1 antibodies, anti-PD-L1 antibodies, combinations thereof) may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Exemplary techniques have been described previously, see, e.g., Remington, The Science and Practice of Pharmacy, 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration can be sterile. This is readily accomplished by, for example, filtration through sterile filtration membranes. Antibodies and antibody compositions intended for therapeutic applications are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid pre-formulation composition containing a homogeneous mixture of a compound of the present disclosure, or a non-toxic pharmaceutically acceptable salt thereof. When referring to these pre-formulation compositions as homogeneous, it is meant that the active ingredient is dispersed evenly throughout the composition so that the composition may be readily subdivided into equally effective unit dosage forms such as tablets, pills and capsules. This solid pre-formulation composition is then subdivided into unit dosage forms of the type described above containing from 0.1 milligrams (mg) to about 500 mg of the active ingredient of the present disclosure. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80, or 85). Compositions with a surface-active agent will conveniently comprise between 0.05% and 5% surface-active agent, and can be between 0.1% and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™, and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%.

The emulsion compositions can be those prepared by mixing an antibody for use in the methods of the instant disclosure (e.g., anti-IL4 antibodies, anti-PD-1 antibodies, anti-PD-L1 antibodies, combinations thereof) with Intralipid™, or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent, or intermittent positive pressure breathing machine. Solution, suspension or powder compositions may be administered, preferably orally or nasally, from devices which deliver the formulation in an appropriate manner.

### G. Antibody Administration

To practice any of the methods disclosed herein, an effective amount of the pharmaceutical composition described above can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized, and lyophilized powder can be nebulized after reconstitution. Alternatively, anti-IL-4 antibodies can be aerosolized using a fluorocarbon formulation and a metered dose inhaler, or inhaled as a lyophilized and milled powder.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a type I diabetes. Alternatively, sustained continuous release formulations of antibodies for use according to the methods of the instant disclosure (e.g., anti-IL-4 antibodies, anti-PD-1 antibodies, anti-PDL-1 antibodies, combinations thereof) may be appropriate. Various formulations and devices for achieving sustained release, for example those provided herein, would be apparent to the skilled artisan and are within the scope of this disclosure.

In one example, dosages for antibodies appropriate for use in the methods of the instant disclosure (e.g., anti-IL-4 antibodies, anti-PD-1 antibodies, anti-PDL-1 antibodies, combinations thereof) may be determined empirically in individuals who have been given one or more administration(s) of the antibody. Individuals are given incremental dosages of the antagonist. To assess efficacy of the antagonist, an indicator of the disease/disorder can be followed.

Generally, for administration of any of the antibodies described herein, an initial candidate dosage can be about 2 mg/kg, however, due to organ specific administration, the dose based on body weight will be misleading. Typical dose ranges in the eye or brain will be in the order of 100 to 50,000 μg, more preferably 500 to 20000 μg, more preferably 1000 to 10000 μg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate type I diabetes, or a symptom thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

In some embodiments, for an adult patient of normal weight, doses ranging from about 0.3 to 5.00 mg/kg may be administered. The particular dosage regimen, e.g., dose, timing and repetition, will depend on the particular individual and that individual's medical history, as well as the properties of the individual agents (such as the half-life of the agent, and other relevant considerations).

For the purpose of the present disclosure, the appropriate dosage of an antibody will depend on the specific antibody employed, the type and severity of the type I diabetes, whether the antibody is administered for preventive or therapeutic purposes, previous therapy, the patient's clinical history and response to the antagonist, and the discretion of the attending physician. In some embodiments, a clinician will administer an antibody, until a dosage is reached that achieves the desired result. Administration of an antibody can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced dose, e.g., either before, during, or after developing type I diabetes.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an antibody is administered via site-specific or targeted local delivery techniques, for example, to a tumor. Examples of site-specific or targeted local delivery techniques include various implantable depot sources of the antibody, or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication Number WO 00/53211 and U.S. Pat. No. 5,981,568.

Targeted delivery of therapeutic compositions containing a polynucleotide, or expression vector can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

Therapeutic compositions containing a polynucleotide encoding the antibodies describe in this disclosure are administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The therapeutic polynucleotides and polypeptides described herein can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

Suitable viral-based vectors for delivery of a desired polynucleotide (e.g., encoding an antibody disclosed herein) and expression in a desired cell are within the scope of this disclosure. Exemplary viral-based vehicles include, but are not limited to, recombinant retroviruses (see, e.g., PCT Publication Numbers: WO 90/07936; WO 94/03622; WO 93/25698; WO 93/25234; WO 93/11230; WO 93/10218; WO 91/02805; U.S. Pat. Nos. 5,219,740 and 4,777,127; GB Patent Number 2,200,651; and EP Patent Numbers 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Numbers: WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication Number: WO 90/11092 and U.S. Pat. No. 5,580,859. Liposomes that can act as gene delivery vehicles are described in U.S. Pat. No. 5,422,120; PCT Publication Numbers: WO 95/13796; WO 94/23697; WO 91/14445; and EP Patent Number: 0524968. Additional approaches are described in Philip, Mol. Cell. Biol. (1994) 14:2411, and in Woffendin, Proc. Natl. Acad. Sci. (1994) 91:1581.

The particular dosage regimen, e.g., dose, timing and repetition, used in the method described herein will depend on the particular subject and that subject's medical history.

### EXAMPLES

### Example 1—Dupilumab with PD-(L)1 Blockade for the Treatment of Relapsed/Refractory Metastatic NSCLC

Inflammation is a hallmark of cancer and chronically inflamed tumor microenvironments have been shown to contribute to the growth and immune-evasion of solid tumors^{1,2}. The tumor microenvironment is often characterized by a complex inflammatory milieu that favors the recruitment and differentiation of suppressive immune cells such as regulatory T cells and macrophages^{3,4}, and dampens the activity of T effector cells and dendritic cells, which are instrumental in presenting tumor-associated antigens to T cells and thereby initiating tumor-directed adaptive immune responses⁵⁻⁷. These suppressive immune cells (e.g., regulatory T cells and macrophages) are skewed towards suppressive phenotypes by anti-inflammatory cytokines present in the tumor microenvironment such as IL-10, TGF-β and Th2 cytokines^{8,9}. IL-4 and IL-13 are the primary mediators of Th2 immune responses and are required to generate immune responses against parasite infections. However, they are also secreted by stressed or dying epithelial cells, and therefore are induced in many solid tumors^{10,11}.

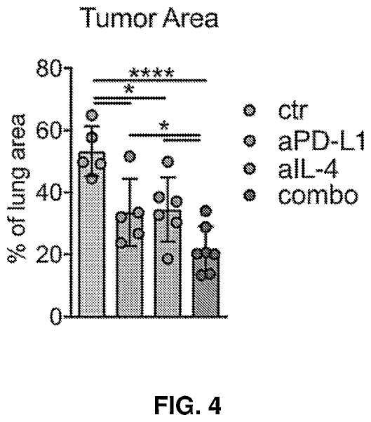

In a mouse model of lung adenocarcinoma (Kras^{G12D}; Tp53^{-/-}), an IL-4-driven regulatory module was recently identified in tumor antigen-charged dendritic cells, indicating IL-4 signaling played an important role in mediating immuno-suppression in this tumor model (FIG. 2A). IL-4 antibody blockade in tumor-bearing mice in vivo led to increased IL-12 production in dendritic cells, a key cytokine produced by mature dendritic cells to activate T cells (FIG. 2B). Subsequently, effector cytokines IFNγ and TNF in CD8⁺ T cells were increased in mice treated with IL-4 blockade (FIG. 2C) and importantly this led to a significant decrease in tumor burden (FIG. 2D) 12. As a monotherapy, IL-4 antibody blockade also significantly decreased the growth of other murine tumor models, such as B16 melanoma, B16 lung metastasis and KP1 lung adenocarcinoma¹³. In the KP1 lung adenocarcinoma model, combination of PD-L1 blockade and IL-4 antibody blockade decreased tumor load compared to either monotherapy alone. See FIG. 4.

The data presented herein provides evidence that blocking Th2 immune mediators, and specifically IL-4 signaling, may be beneficial for cancer patients by activating dendritic cells and subsequent T effector cells generating a robust immune response against tumor antigens. In addition, it is hypothesized that Th2 skewing of the tumor microenvironment might impair the patients' response to immunotherapy. Therefore, a clinical trial combining IL-4 blockade with PD-1/PD-L1 blockade is very promising with respect to both the response rate as well as the magnitude of response.

Dupilumab is a fully human monoclonal antibody to the interleukin-4 (IL-4) receptor alpha subunit which disrupts signaling through receptors for both IL-4 and IL-13, two cytokines known to be classic Th-2 polarizing cytokines. Dupilumab is FDA-approved for the treatment of moderate to severe asthma, atopic dermatitis and for patients with chronic rhinosinusitis with nasal polyps based on promising data in these diseases where few good alternatives exist¹⁴⁻¹⁹. This treatment is very well tolerated in these atopic patients, with the most common side effect being injection site reactions occurring in less than 20% of people; these reactions are typically low grade. The use of dupilumab has yet to be explored specifically in patients to target pro-tumorigenic Th2 polarization.

PD-(L)1 blocking agents are used in the treatment of nearly all patients with NSCLC who lack a targetable driver mutation who are being treated for metastatic disease, or as an adjuvant following concurrent chemoradiotherapy for patients with unresectable locally advanced disease²⁰⁻²³. Incorporation of PD-(L)1 blocking agents into the treatment paradigm for NSCLC has significantly improved overall survival for patients with NSCLC, though the majority of patients eventually succumb to their disease. For patients with metastatic disease the current treatment paradigm dictates the use of immunotherapy—most commonly pembrolizumab (KEYTRUDA®, an anti-PD-1 antibody), though nivolumab (e.g., OPDIVO®, an anti-PD-1 antibody) in conjunction with ipilimumab (e.g., YERVOY®, an anti-CTLA-4 antibody) has also been FDA approved—until progression of disease or intolerance of therapy for up to two years, while patients receiving adjuvant therapy following concurrent chemoradiation typically receive up to 12 months of the anti-PD-L1 antibody durvalumab (e.g., IMFINZI®, an anti-PD-L1 antibody)²⁰⁻²³. PD-L1 expression, tumor type, and patient performance status typically dictate whether chemotherapy is incorporated into the induction and maintenance therapy for patients. In general PD-1 antibodies are thought to be moderately more efficacious and moderately more toxic, but overall the agents are comparable across trials^{24,25}.

There is clinical rationale for the combination of these therapies, particularly cancers that have the potential to respond, but most commonly are either refractory or develop resistance to PD-1/PD-L1 blocking antibodies (e.g. head and neck squamous cell carcinoma (HNSCC), non-small cell lung cancer (NSCLC), hepatocellular carcinoma (HCC), and mesothelioma). Lack of response, or loss-of-response to antibodies blocking the PD-1/PD-L1 axis may represent lack of immunogenicity, however, based on previous data, it is proposed that a highly immunosuppressive Th-2 polarized tumor immune microenvironment promotes resistance to effective immune recognition.

### Study Design

Patients who have progressed on PD-(L)1 targeted therapies will be enrolled; optimally PD-(L) 1 targeting agents will be the immediate prior therapy, though up to one line of intervening anti-cancer therapy is permitted. Patients must be candidates for standard of care (SOC) PD-1 or PD-L1 therapy. Patients will receive 3 treatments with dupilumab as part of this clinical trial, the first to be given in conjunction with standard PD-(L)1 targeted therapy infusion. Dupilumab is administered as a subcutaneous injection. Patients enrolled in this trial upon documented (radiographic or clinical) progression of disease will undergo core needle biopsy and specified blood collection prior to starting therapy, and they will undergo repeat core needle biopsy of their cancer 4 weeks after receipt of their first dupilumab injection. Following their final dupilumab injection patients will undergo repeat imaging, which will define response to therapy.

While anecdotal experience of using Dupilumab in cancer patients suggests there will be no synergistic toxicity concerns, this combination has not formally been prospectively investigated in the setting of a clinical trial. As such there will be a Phase 1b run-in clinical trial, in which first 3 patients will be enrolled and the first cycle (21 days) constitutes the DLT window. If there are 2 or more patients experiencing a DLT, the trial will be halted and the treatment plan discussed with the DSMC. If 0 or 1 patient experience a DLT, 3 more patients (total of 6 patients) will be entered. If 2 or more patients experience a DLT the trial will be halted and the treatment plan discussed with the DSMC. If at most 1 patient out of 6 patients experiences a DLT, the Phase 2 portion of the trial will open. The six patients from the Phase 1b portion will be evaluable as part of the Phase 2 cohort of 21 patients. The 21 patient cohort will follow a two-stage minimax design, with a stopping rule for futility.

### Study Objectives

### Primary Objective

The primary objective of the Phase 1b portion of the study is to determine the safety and tolerability of combined treatment of Dupilumab and PD-(L)1 targeted therapy in patients with relapsed or refractory NSCLC.

The primary objective of the Phase 2 trial is to assess efficacy of the addition of dupilumab to patients with relapsed or refractory NSCLC who have progressed on prior PD-(L)1 targeted therapy, by measuring overall response rate by imaging.

### Secondary Objectives

The clinical secondary objectives of this trial include:

To further assess toxicity of combined treatment of dupilumab and PD-(L)1 targeted therapy in patients with relapsed or refractory NSCLC in the expanded Phase 2 portion of the study.
Best overall response (BORR).
Progression-free survival (PFS).
Overall survival (OS).
Duration of response (DOR).

### Exploratory Scientific Objectives

The non-clinical objective of this trial is to document the evolution of a patient's tumor microenvironment (TME), comparing pre-treatment biopsy and blood with tumor and blood taken from the patient at time of surgical resection. This will include histologic, immunologic, genetic, and radiomic characterization of the tumor both in bulk and at the single cell level. Analysis of blood samples will include, but is not limited to, mass cytometry (CyTOF) analysis of peripheral blood mononuclear cells, as well as seromic analysis using ELISA and/or O-link technologies. Tissue, when available, will be analyzed transcriptomically (through bulk and/or single-cell RNA sequencing based on the amount of tissue available) and proteomically though multiplex immunohistochemistry and/or through CyTOF of dissociated cells. Whole exome sequencing may also be performed to aid in detection of mutations that may result in neoantigens. Additionally, circulating tumor DNA (ctDNA) will be assessed at multiple time-points to assess potential to detect tumor response and/or recurrence using this novel assay, and TCR sequencing will be performed on tumor and matched blood samples. Study Endpoints

### Primary Endpoint

The primary endpoint for the Phase 1b portion of the study is dose limiting toxicity (DLTs) as defined in section 9.3, based on the NCI Common Toxicity Criteria for Adverse Events (CTCAE), version 5.0.

The primary endpoint of the Phase 2 trial is overall response rate by imaging at time of first repeat imaging (˜9w from the start of therapy) using standard response criteria (RECIST v1.1²⁶). ORR is defined as the combined percent of the patients experiencing a partial response (PR) or a complete response (CR) at time of first reimaging.

### Secondary Endpoints

The clinical secondary endpoints of this trial include:

Safety and Tolerability: For the Phase 2 trial, toxicity will be assessed according to the NCI Common Toxicity Criteria for Adverse Events (CTCAE), version 5.0.

Best overall response (BORR) will be a combined percent of the patients experiencing a partial response (PR) or a complete response (CR) at any point within the first year from the initiation of therapy, or until the documented progression of disease or start of a new anti-cancer therapy.

Progression-free survival (PFS) is defined as the time in days from the first administration of dupilumab until documented progression of disease on imaging or death

Overall survival (OS) is defined as the time in days from the first administration of dupilumab until documented death from any cause.

Duration of response (DOR) is defined as the time from which a patient achieves either a PR or a CR until subsequent progression of disease is documented radiographically or clinically

### Exploratory Endpoints

The primary non-clinical endpoint of this trial is the creation of dynamic atlases documenting the evolution of a patient's TME, comparing pre-treatment biopsy and blood with subsequent biopsy and blood collection. The formation of this atlas involves histologic, immunologic, genetic and radiomic characterization of the tumor both in bulk and at the single cell level, across time (from pre-treatment biopsy to biopsy upon recurrence) and across patients, and potentially even organ-specific cohorts. ctDNA will be measured at multiple time points during treatment using an investigational available assay, and the ability of this assay to detect recurrence ahead of radiographic evidence of recurrence measured.

### Patient Eligibility

### Inclusion Criteria

Patients must have a pathologically confirmed diagnosis of NSCLC
Patients must have progressed (clinically or radiographically) on or following prior therapy with a PD-1 or PD-L1 targeted antibody
Patients may have only 0 or 1 intervening lines of therapy from the prior PD-(L)1 blocking therapy
Patient must be willing and able to provide blood samples (12 green-top tubes, roughly 100 mL) at the time points indicated in the Study Calendar.
Patient must be willing and able to have core needle biopsies, or forceps biopsies if clinically feasible by (Goal 3-6 biopsies, final number to be determined by the interventionalist performing the procedure as safe) of tumor prior to initiation of dupilumab and at the on-treatment time point.
Age ≥18 years.
ECOG 0-2. The exception will be patients carrying long term disability (such as cerebral palsy) where the disability is not acute nor progressive, and unlikely to significantly affect their response to therapy.
Women of child-bearing potential and men must agree to use adequate contraception prior to study entry, for the duration of study participation, and for 3 months following completion of therapy. Should a woman become pregnant or suspect she is pregnant while participating in this study, she should inform her treating physician immediately. A female of child-bearing potential is any woman (regardless of sexual orientation, having undergone a tubal ligation, or remaining celibate by choice) who meets the following criteria:
Has not undergone a hysterectomy or bilateral oophorectomy; or.
Has not been naturally postmenopausal for at least 12 consecutive months
Ability to understand and the willingness to sign a written informed consent.
Adequate organ and marrow function as defined below in Table 1:

TABLE 1

| System | Laboratory Value |
|---|---|
| Hematologic | |
| Absolute neutrophil count (ANC) | ≥1,000/mcL |
| Platelets | ≥75,000/mcL |
| Hemoglobin | ≥9 g/dL |
| Renal* | |
| Serum creatinine OR | ≤1.5 × upper limit of normal (ULN) |
| Measured or calculateda creatinine clearance | OR |
| (GFR can also be used in place of creatinine or | ≥60 mL/min for patient with creatinine levels > 1.5 × |
| CrCl) | institutional ULN |
| Hepatic* | |
| Serum total bilirubin | ≤1.5 × ULN OR |
| | Direct bilirubin ≤ ULN for patients with total bilirubin levels > |
| | 1.5 ULN |
| | ≤3 × ULN for patients with liver metastases |
| AST and ALT | ≤2.5 × ULN OR |
| | ≤5 × ULN for patients with liver metastases |
| Albumin | >2.5 mg/dL |
| Coagulation* | |
| International Normalized Ratio (INR) or | ≤1.5 × ULN unless patient is receiving anticoagulant therapy |
| Prothrombin Time (PT) | as long as PT is within therapeutic range of intended use of |
| | anticoagulants |
| Activated Partial Thromboplastin Time (aPTT) | ≤1.5 × ULN unless patient is receiving anticoagulant therapy |
| | as long as PTT is within therapeutic range of intended use of |
| | anticoagulants |

a Creatinine clearance should be calculated per institutional standard.
*If laboratory criteria are not met due to what the investigator determines to be a biologic cause (e.g. Gilbert's syndrome causing elevated bilirubin or excessive muscle mass affecting creatinine) or drug-related cause (e.g. elevating in transaminases due to HAART therapy, elevated INR due to anticoagulation) then the lab values will not be used to exclude patient from this trial. This determination will be made by PI.

### Exclusion Criteria:

Patients who have had chemotherapy or radiotherapy within 14 days from start of therapy. Washout for palliative radiotherapy is 14 days.
Patients may not be receiving any other investigational agents.
Uncontrolled intercurrent illness including, but not limited to, ongoing or active infection requiring antibiotics (exception is a brief (≤10 days) course of antibiotics to be completed before initiation of treatment), symptomatic congestive heart failure, unstable angina pectoris, or psychiatric illness/social situations that would limit compliance with study requirements.
Patients must not be pregnant or nursing due to the potential for congenital abnormalities and the potential of this regimen to harm nursing infants.
Has a diagnosis of immunodeficiency or is receiving systemic steroid therapy or any other form of immunosuppressive therapy within 7 days prior to the first dose of trial treatment. Patients on chronic steroids (more than 4 weeks at stable dose) equivalent to ≤10 mg prednisone will not be excluded.
Has active autoimmune disease that has required systemic treatment in the past 1 year (i.e. with use of disease modifying agents, corticosteroids or immunosuppressive drugs). Replacement therapy (e.g. thyroxine, insulin, or physiologic corticosteroid replacement therapy for adrenal or pituitary insufficiency, etc.) is acceptable.
Has a history or current evidence of any condition, therapy, or laboratory abnormality that might confound the results of the trial, interfere with the patient's participation for the full duration of the trial, or is not in the best interest of the patient to participate, in the opinion of the treating Investigator.
HIV positive with detectable viral load, or anyone not on stable anti-viral (HAART) regimen, or with <350 CD4⁺ T cells/microliter in the peripheral blood.
Has known active Hepatitis B (e.g., HBV detected by PCR or active Hepatitis C (e.g., HCV RNA [qualitative] is detected). Patients with hepatitis B (HepBsAg+) who have controlled infection (serum hepatitis B virus DNA PCR that is below the limit of detection AND receiving anti-viral therapy for hepatitis B) are permitted. Patients with controlled infections must undergo periodic monitoring of HBV DNA. Patients must remain on anti-viral therapy for at least 6 months beyond the last dose of investigational study drug.
History of allogeneic hematopoietic cell transplantation or solid organ transplantation.
Receipt of a live vaccine within 30 days of planned start of study medication
Documented allergic or hypersensitivity response to any protein therapeutics (e.g., recombinant proteins, vaccines, intravenous immune globulins, monoclonal antibodies, receptor traps) Principle investigator believes that for one or multiple reasons the patient will be unable to comply with all study visits, or if they believe the trial is not clinically in the best interest of the patient.
History of irAE in response to prior immunotherapy that has not improved to a Grade 0 or 1; this does not include endocrinopathies which can be treated with hormone replacement therapy.
History of interstitial lung disease (e.g., idiopathic pulmonary fibrosis, organizing pneumonia) or active, noninfectious pneumonitis attributed to prior use of cancer immunotherapy that required immune-suppressive doses of glucocorticoids to assist with management. A history of radiation pneumonitis in the radiation field is permitted.

### Treatment Plan

TABLE 2
Treatment Dosage and Administration
REGIMEN DESCRIPTION

| Agent | Dose | Route | Schedule |
|---|---|---|---|
| Dupilumab | 300 mg | SQ | 2 injections (600 mg total) |
| | | | given D 1, |
| | | | 1 injection (300 mg) given |
| | | | Day 22 and 43 |
| Anti-PD-(L)1 | Continue as | Continue as | Continue as |
| therapy | per SOC | per SOC | per SOC |

### Dose Selection and Schedule

Patients will receive dupilumab (subcutaneously (SQ) every 3 weeks for 3 doses. 600 mg on D1 and 300 mg on subsequent doses is a standard dose that has been tested in multiple clinical trials, though that more frequent dosing schedule, however, it is scheduled to be dosed every 3 weeks in this trial to coincide with the most common dosing schedule for patients receiving maintenance pembrolizumab, who will constitute the majority of patients enrolled in this clinical trial^{27,28}. This dosing schedule will limit extraneous clinical visits. Patients will receive SOC PD-(L)1 antibodies at a dose and dosing schedule defined by treating investigator to constitute SOC. All treatment will be administered as an outpatient at the Ruttenberg Treatment Center (Mount Sinai). Patients will receive the dupilumab before administration of their PD-(L)1 antibody, to allow for observation following the dupilumab.

### Toxicities and Dosing Delays

Toxicity will be assessed according to the NCI Common Toxicity Criteria for Adverse Events (CTCAE), version 5.0.

### Definition of Dose-Limiting Toxicity (DLT) and Maximum Tolerated Dose (MTD)

Dose delays will be permitted as outlined herein. Subjects who experience a DLT will be discontinued from treatment. The first cycle (21 days) constitutes the DLT window.

The occurrence of any of the following toxicities during the DLT window, if assessed by the Investigator to be related, probably related, or possibly related to dupilumab and PD-(L)1 administration will be considered a DLT:
Grade 4 non-hematologic toxicity (not laboratory)
Grade 4 hematologic toxicity lasting ≥7 days
Any non-hematologic AE≥Grade 3 in severity should be considered a DLT, with the following exceptions: Grade 3 fatigue lasting ≤5 days; Grade 3 nausea, vomiting, or diarrhea lasting ≤5 days. Grade 3 rash without use of corticosteroids or anti-inflammatory agents per SOC; Grade 4 fever and Grade 4 flu-like symptoms lasting ≤48 hours.
Any new Grade 3 or Grade 4 non-hematologic laboratory abnormality, if
   medical intervention is required, or
   the abnormality leads to hospitalization, or
   the abnormality persists for >1 week
Febrile neutropenia Grade 3 or Grade 4
Any elevated AST or ALT>3×ULN and concurrent total bilirubin >2×ULN without initial findings of cholestasis or biliary dilatation on imaging (elevated ALP, e.g., findings consistent with Hy's law or FDA definition of potential drug-induced liver injury [p-DILI]). Note that this specific category of DLT uses ULN rather than CTCAE grade for definition.
Any ≥Grade 2 immune-mediated uveitis
Grade 5 toxicity

While rules for adjudicating DLTs are specified here, DLTs, AEs, and irAEs may be defined after consultation with sponsor and principal investigator based on emerging data from the use of checkpoint blockade therapy. irAEs are defined as AEs of inflammatory immune nature in the absence of a clear alternative etiology such as infection.

Patients that experience DLT requiring discontinuation from the study will enter the monitoring phase of the study with regular scheduled imaging and symptom assessment as per SOC, and their symptoms and disease course will be recorded.

### Dose Modification/Delays

AEs (both non-serious and serious) associated with dupilumab exposure may represent an immunologic etiology. These AEs may occur shortly after the first dose or several months after the last dose of treatment. Dupilumab and PD-(L)1 antibodies must both be withheld for drug-related toxicities and severe or life-threatening AEs as per Table below.

TABLE 3
Dupilumab and SOC PD-L(1) dose modification or discontinuation based on specific irAE grade

| | Hold | | |
|---|---|---|---|
| | Treatment | Timing for Restarting | |
| Toxicity | For Grade | Treatment | Discontinue Patient |
| Diarrhea/Colitis | 2 | Toxicity resolves to | Toxicity does not resolve within 12 weeks of last |
| | | Grade 0-1. | dose or inability to reduce corticosteroid to 10 mg |
| | | | or less of prednisone or equivalent per day within |
| | | | 12 weeks. |
| | 3-4 | Permanently discontinue | Permanently discontinue |
| AST, ALT, or | 2 | Toxicity resolves to | Toxicity does not resolve within 12 weeks of last |
| Increased | | Grade 0-1 | dose. |
| Bilirubin | 3-4 | Permanently discontinue | Permanently discontinue |
| | | (see exception below)¹ | |
| Type 1 diabetes | T1DM or | Hold PD-(L)1 agent and | Resume PD-(L)1 and dupilumab therapy when |
| mellitus (if new | 3-4 | dupilumab for new onset | patients are clinically and metabolically stable. |
| onset) or | | Type 1 diabetes mellitus | |
| Hyperglycemia | | or Grade 3-4 | |
| | | hyperglycemia | |
| | | associated with evidence | |
| | | of beta cell failure. | |
| Hypophysitis | 2-3 | Toxicity resolves to | Toxicity does not resolve within 12 weeks of last |
| | | Grade 0-1 | dose or inability to reduce corticosteroid to 10 mg |
| | | | or less of prednisone or equivalent per day within |
| | | | 12 weeks. |
| | 4 | Permanently discontinue | Permanently discontinue |
| Hyperthyroidism | 3 | Toxicity resolves to | Toxicity does not resolve within 12 weeks of last |
| | | Grade 0-1 | dose or inability to reduce corticosteroid to 10 mg |
| | | | or less of prednisone or equivalent per day within |
| | | | 12 weeks. |
| | 4 | Permanently discontinue | Permanently discontinue |
| Hypothyroidism | 2-4 | Therapy with PD-(L)1 | Therapy with PD-(L)1 agent and dupilumab can be |
| | | agent and dupilumab can | continued while treatment for the thyroid disorder is |
| | | be continued while | instituted. |
| | | treatment for the thyroid | |
| | | disorder is instituted | |
| Infusion | 3-4 | Permanently discontinue | Permanently discontinue |
| Reaction | | | |
| Pneumonitis | 2 | Toxicity resolves to | Toxicity does not resolve within 12 weeks of last |
| | | Grade 0-1 | dose or inability to reduce corticosteroid to 10 mg |
| | | | or less of prednisone or equivalent per day within |
| | | | 12 weeks. |
| | 3-4 | Permanently discontinue | Permanently discontinue |
| Renal Failure or | 2 | Toxicity resolves to | Toxicity does not resolve within 12 weeks of last |
| Nephritis | | Grade 0-1 | dose or inability to reduce corticosteroid to 10 mg |
| | | | or less of prednisone or equivalent per day within |
| | | | 12 weeks. |
| | 3-4 | Permanently discontinue | Permanently discontinue |
| All Other Drug- | 3 or Severe | Toxicity resolves to | Toxicity does not resolve within 12 weeks of last |
| Related | | Grade 0-1 | dose or inability to reduce corticosteroid to 10 mg |
| Toxicity² | | | or less of prednisone or equivalent per day within |
| | | | 12 weeks. |
| | 4 | Permanently discontinue | Permanently discontinue |

Note:
Permanently discontinue for any severe or Grade 3 drug-related AE that recurs or any life-threatening event.
¹For patients with liver metastases who begin treatment with Grade 2 AST or ALT, if AST or ALT increases by greater than or equal to 50% relative to baseline and lasts for at least 1 week then patients should be discontinued.
²Patients with intolerable or persistent Grade 2 drug-related AE may hold study medication at physician discretion. Permanently discontinue study drug for persistent Grade 2 adverse reactions for which treatment with study drug has been held, that do not recover to Grade 0-1 within 12 weeks of the last dose.

### Timing of Dose Administration

Study components will be administered as laid out in Study Calendar. PD-(L)1 agent and dupilumab should be administered on the same day, and dupilumab should be dosed every 21±5 days as outlined above, while PD-(L)1 agent should be administered as determined to be SOC by treating investigator. Every attempt should be made to perform all assessments and administer all treatments as per the calendar below, but may be adjusted by the treating physician based on what is determined to be in the best interest of the patient.

### Duration of Follow-Up

Patients will be followed for at least 30 days for AE monitoring following last dose of dupilumab received. Patients who do not have confirmed progression of disease or intolerance to therapy during this period will continue PD-(L)1 therapy as pre SOC, and clinical status and survival will be monitored as per SOC. The study team will monitor the status of the patient through chart review or by telephone should the patient not continue to follow with a physician at Mount Sinai, for up to 5 years or until death, withdrawal of consent, or the end of the study, whichever occurs earlier.

### Patient Replacement

Patients enrolled and withdrawn from study any time prior to first dose of dupilumab will not be considered evaluable for assessment and will be replaced with another patient. All patients who have received a first dose of PD-(L)1 agent and dupilumab (having received both treatments in full on C1D1) will be included in an intention-to-treat analysis.

### Concomitant Medications/Vaccinations (Allowed & Prohibited)

Medications or vaccinations specifically prohibited in the exclusion criteria are not allowed during the ongoing trial. If there is a clinical indication for one of these or other medications or vaccinations specifically prohibited during the trial, discontinuation from trial therapy may be required. The Investigator should discuss any questions regarding this with the Study PI and Sponsor. The final decision on any supportive therapy or vaccination rests with the Investigator and/or the patient's primary physician.

### Acceptable Concomitant Medications

All treatments that the Investigator considers necessary for a patient's welfare may be administered at the discretion of the Investigator in keeping with the community standards of medical care. All concomitant medication will be recorded in the medical record, including all prescription, over-the-counter, herbal supplements, and intravenous medications and fluids. If changes occur during the trial period, documentation of drug dosage, frequency, route, and date may also be included in the medical record.

All concomitant medications received within 28 days before the first dose of trial treatment and 30 days after the last dose of trial treatment should be recorded.

### Prohibited Concomitant Medications

Patients are prohibited from receiving the following therapies during the Screening and during the 9 weeks of treatment on this trial:
Antineoplastic systemic chemotherapy or biological therapy
Immunotherapy not specified in this protocol
Chemotherapy not specified in this protocol.
RT therapy, except for palliative purposes
Live vaccines within 30 days prior to the first dose of trial treatment and while participating in the trial. Examples of live vaccines include, but are not limited to, the following: measles, mumps, rubella, varicella/zoster, yellow fever, rabies, BCG, and typhoid vaccine.
Systemic glucocorticoids for any purpose other than those used to modulate symptoms from an AE of suspected immunologic etiology.

Patients who, in the assessment by the Investigator, require the use of any of the aforementioned prohibited treatments for clinical management should be removed from the trial. Patients may receive other medications that the Investigator deems to be medically necessary.

### Study Procedures

### Screening/Baseline Procedures

Assessments performed exclusively to determine eligibility for this study will be done only after obtaining informed consent. Assessments performed for clinical indications (not exclusively to determine study eligibility) may be used for baseline values even if the studies were done before informed consent was obtained.

All screening procedures must be performed within 28 days prior to registration unless otherwise stated. The screening procedures include:
Informed Consent
Medical history
Review subject eligibility criteria
Review previous and concomitant medications
Physical exam including vital signs, height and weight
Eastern Cooperative Oncology Group (ECOG) Performance Scale
Baseline adverse event assessment

### Laboratory Procedures/Assessments

Pregnancy Test: A serum or urine pregnancy testing is required within 24 hours of study enrollment for female patients who have menstruated within the preceding 48 months.

Laboratory testing at screening: CBC w/differential, CMP, magnesium, TSH (with reflex Free T4), PT/PTT.

Laboratory testing prior to each dose study agent (for 3 cycles): Within 72 hours prior to first treatment dose labs must be drawn which include CBC w/differential, CMP, magnesium, TSH (with reflex Free T4). These laboratories are SOC for immunotherapy administration at Mount Sinai Hospital.

Details regarding timing of specific laboratory procedures/assessments to be performed in this trial are provided in Table 4, below.

TABLE 4
Laboratory Tests (Hematology/chemistry/urinalysis/other)

| Hematology | Chemistry | Urinalysis | Other |
|---|---|---|---|
| Hematocrit | Albumin | Urine pregnancy test† | Serum β-human |
| | | | chorionic |
| | | | gonadotropin(β-hCG)† |
| Hemoglobin | Alkaline phosphatase | | PT (INR) |
| Platelet count | Alanine aminotransferase (ALT) | | aPTT |
| WBC (total and differential) | Aspartate aminotransferase (AST) | | Reflex free thyroxine |
| | | | (T4) |
| Red Blood Cell Count | Carbon Dioxide (CO₂ or | | Thyroid stimulating |
| | bicarbonate)‡ | | hormone (TSH) |
| Absolute Neutrophil Count | Calcium | | Correlative studies |
| | | | (blood) |
| Absolute Lymphocyte | Chloride | | |
| Count | Glucose | | |
| | Potassium | | |
| | Sodium | | |
| | Magnesium | | |
| | Total Bilirubin | | |
| | Direct Bilirubin (If total bilirubin | | |
| | is above upper limit normal) | | |
| | Total protein | | |
| | Blood Urea Nitrogen | | |

†For women of childbearing potential, specifically anyone who has menstruated within the 48 months before enrollment. If urine pregnancy not confirmed negative, serum pregnancy test required.
‡If considered standard of care in the region.

Laboratory tests for screening should be performed within 28 days prior to the first dose of treatment, and CBC, CMP, magnesium, and TFTs before each dose of dupilumab administration, as peer SOC prior to all administrations of PD-(L)1 agents. At ISMMS the CMP laboratory panel includes all components listed above in “chemistry” other than Direct Bilirubin and Magnesium. Results must be reviewed by the Investigator or qualified designee and found to be acceptable prior to each dose of trial treatment (with the exception of TFTs which will often take longer, and are to be checked regularly for trends suggestive of thyroiditis).

### Procedures During Treatment

PD-(L)1 agent and Dupilumab Infusion: Patients will receive their first dose of PD-(L)1 agent and dupilumab on day 1 of the trial (D1), their second dose of dupilumab on D22±5 days, and 3^{rd} dose on D43 (±5 days). The PD-(L)1 agent will be administered as per SOC dosing schedule, at the discretion of the treating investigator.

### Imaging

Imaging should have been performed during the 28 days prior to initiation of study therapy, though if there is a delay in dosing (C1D1) of less than 2 weeks for any clinical or logistical reason repeat baseline imaging will not be mandates, so as to limit excessive radiation exposure. Subsequent SOC imaging will occur at 9 weeks (D63±9 days), this imaging will serve to define the primary endpoint. Subsequent imaging schedule, should a patient continue with PD-(L)1 therapy should be done at whatever SOC interval is defined by the treating investigator.

### Biopsy (Pre-Treatment)

Patients will undergo core needle or forceps/excisional biopsy during the screening period and at 4 weeks (D29±10 days). The target collection is 3-6 biopsies, final number to be determined as safe by the pulmonologist and/or radiologist performing the procedure. This tissue will be evaluated by pathology to confirm diagnosis, and remaining tissue will be transported to the Human Immune Monitoring Core (HIMC).

### Peripheral Blood

Patients will have peripheral blood drawn (12 green-top tubes, roughly 100 mL) which will be processed by the Human Immune Monitoring Core, fresh blood may be used for CyTOF analysis, and PBMCs and plasma will be cryopreserved for future investigations. Blood will be drawn:
At screening, or before treatment on D1
D4±3 days (goal is to collect on D4)
D8±3 days
D15±3 days
D22±5 days (on day of second dupilumab injection)
D43±5 days (on day of third dupilumab injection)

### Study Calendar

D1 represents the first day of PD-(L)1 agent and dupilumab.

| | | | D4, D8, | | | |
|---|---|---|---|---|---|---|
| | Screening | D1 | D15 (±3d) | D22 ± 5d | D43 ± 5d | D63 ± 9d |
| Informed Consent | X | | | | | |
| Medical history | X | | | | | |
| Physical exam | X | X | | X | X | |
| ECOG determination | X | X | | X | X | |
| Toxicity Evaluations | X | X | | X | X | |
| Imaging1 | X | | | | | X |
| Standard Labs3 | X | X | | X | X | |
| Screening Labs4 | X | | | | | |
| Biopsy5 | X | | | X | | |
| Research Blood6 | X | X | X | X | X | |
| ctDNA Tube2 | X | | X | X | | |

1Imaging to occur within 28d of D1, and then at D63 ± 9D. Subsequent imaging as determined to be SOC per investigator.
2A specialized tube of blood dedicated to preservation of plasma ctDNA will be drawn alongside research blood at each time point listed above.
3Standard labs (SOC) include CBC w/differential, CMP, Magnesium, and TSH (with reflex free T4) and cortisol.
4Screening labs include the standard labs, as well as pregnancy test if needed (urine or serum), PT/PTT (or PT/aPTT).
5Biopsies to include (target) 3-6 core needle biopsies of tumor, or as determined as safe by interventionalist and/or radiologist. Second biopsy should be performed on D29 ± 10d
6As described above, 100 mL (roughly 12 large green top heparinized tubes) of research blood to be collected at regular intervals through D43.

Removal of Subjects from Study

Patients can be taken off the study treatment and/or study at any time at their own request, or they may be withdrawn at the discretion of the investigator for safety, behavioral or administrative reasons.

### Measurement of Effect

### Response Criteria

Radiographic response. ORR at initial imaging time point is the primary endpoint of this clinical trial. Responses to neoadjuvant therapy (based on pre-surgery imaging compared to pre-treatment imaging) will be defined using the RECIST v1.1²⁶:
Complete Response (CR): Disappearance of all lesions.
Partial Response (PR): ≥30% decrease in tumor burden compared with baseline.
Progressive Disease (PD): At least 20% increase in tumor burden compared with nadir (at any single time point).
Stable Disease (SD): 30% decrease in tumor burden compared with baseline cannot be established nor 20% increase compared with nadir.
BORR is defined as the combined percent of the patients experiencing a partial response (PR) or a complete response (CR) at any point within the first year from the initiation of therapy, or until the documented progression of disease or start of a new anti-cancer therapy.
Duration of response (DOR) is defined as the time from which a patient achieves either a PR or a CR until subsequent progression of disease is documented radiographically or clinically.

### Safety/Tolerability

Analyses will be performed for all patients having received at least one dose of study drug. The study will use the NCI CTCAE version 5.0 for reporting of non-hematologic adverse events (ctep.cancer.gov) and modified criteria for hematologic adverse events.

### PFS

PFS is calculated based on the time from treatment initiation until the time of radiographic evidence of progression of disease

### OS

OS is defined as the time from treatment initiation until the time of death from any cause.

### Adverse Events

### Definition of Adverse Event

An adverse event (AE) is any untoward medical occurrence in a patient receiving study treatment and which does not necessarily have a causal relationship with this treatment. An AE can therefore be any unfavorable and unintended sign (including an abnormal laboratory finding), symptom, or disease temporally associated with the use of an experimental intervention, whether or not related to the intervention.

### Adverse Event Monitoring

The Investigator or qualified designee will assess each subject to evaluate for potential new or worsening AEs as specified in the Study Calendar (as toxicity evaluations). At each of these toxicity evaluation periods patients will have a history and physical performed, as well as blood analyses (CBC w/differential, CMP, magnesium, TSH [with reflex free T4] and cortisol) to screen for any potential irAE. Following the third cycle patients will continue SOC administration of PD-(L)1 administration at the discretion of the treating investigator, and irAE will be monitored and recorded in the medical record as per SOC.

Each AE of unknown etiology associated with PD-(L)1 agent exposure should be evaluated to determine if it is possibly an event of clinical interest of a potentially irAEs.

### Supportive Care Guidelines

Any diagnostic or therapeutic procedures needed to provide optimal medical care to patients while on study should be performed.

Patients should receive appropriate supportive care measures as deemed necessary by the treating Investigator. It may be necessary to perform conditional procedures such as bronchoscopy, endoscopy, or skin biopsy and/or photography as part of evaluation of the event.

Supportive care measures for the management of AEs with potential immunologic etiology should be provided as per ASCO²⁹ or SITC³⁰ guidelines.

### Serious Adverse Events

A “serious” adverse event is defined in regulatory terminology as any untoward medical occurrence that:

Results in death: If death results from (progression of) the disease, the disease should be reported as event (SAE) itself.
Is life-threatening: The patient was at risk of death at the time of the event; it does not refer to an event that hypothetically might have caused death if it were more severe.
Requires in-patient hospitalization or prolongation of existing hospitalization for ≥24 hours.
Results in persistent or significant disability or incapacity.
Is a congenital anomaly/birth defect.
Is an important medical event. Any event that does not meet the above criteria, but that in the judgment of the investigator jeopardizes the patient, may be considered for reporting as a serious adverse event. The event may require medical or surgical intervention to prevent one of the outcomes listed in the definition of “Serious Adverse Event”.
   For example: allergic bronchospasm requiring intensive treatment in an emergency room or at home; convulsions that may not result in hospitalization; development of drug abuse or drug dependency.

### Stopping Rules

There will be continuous monitoring of the dose limiting toxicities (DLTs) following treatment with the combination of dupilumab and PD-(L)1 blockade. The first cycle (21 days) constitutes the DLT window. Accrual will be halted if there is sufficient evidence that the DLT rate exceeds the acceptable target rate of 20% by more than 20%. If the cumulative number of patients experiencing a DLT is greater than the associated boundary value br listed in the table below, among the k patients enrolled, then accrual will be halted for safety considerations. There will additionally be a pre-planned analysis of the endpoints after the first 10 patients.

| # of Patients | 6 | 7-9 | 10-12 | 13-15 | 16-18 | 18-21 |
|---|---|---|---|---|---|---|
| Boundary | 1 | 3 | 4 | 5 | 6 | 7 |

| | True Toxicity Rate | | | | |
|---|---|---|---|---|---|
| | 0.20 | 0.25 | 0.30 | 0.35 | 0.40 |
| Probability of Early Stopping | 0.36 | 0.51 | 0.65 | 0.77 | 0.87 |

Specifically, if more than 1 of the first 6 patients, or more than 3 of the first 9 patients, or more than 4 of the first 12 patients, or more than 5 of the first 15 patients, or more than 6 of the first 18 patients or more than 7 of the first 21 patients experience DLTs within the first cycle, the trial will be halted for safety considerations.

The operating characteristics of this stopping rule are given in the above table.

Using these boundaries, if the true toxicity rate is 20%, 25%, 30%, 35% or 40%, the probability of stopping the trial early is 0.36, 0.51, 0.65, 0.77, and 0.87, respectively. This stopping rule was computed using the method of Ivanova, Qaquish, and Schell³¹ and using the following parameters; and using the bodycross function from the ClinFun package in R:
r=c(1,3,4,5,6,7)
n=c(6,9,12,15,18,21)
ptox=seq(0.20,0.40,by=0.05)
bdrycross.prob(n,r,ptox)

### Correlatives/Special Studies

Some of the assays that will be performed on the tissue are listed below, and given the rapid development of technologies in this field, preserved tissue from this trial may be analyzed using additional platforms in the years to come.

Time-of-flight Mass Cytometry (CyTOF): CyTOF allows the simultaneous analysis of over 50 parameters on peripheral blood or tumor cell suspensions without need for compensations. A novel mass cytometry barcoding strategy has been established that allows multiple samples to be pooled together, processed, and analyzed as a single sample, thereby reducing batch effects and dramatically enhancing the robustness and reproducibility. This technology has been applied to map human cancer lesions and identified unique immune changes that occur in early stages tumor lesions⁴. This will be used to phenotype the tumor immune infiltrate as well as the blood, and the phenotypic changes that occur with neoadjuvant therapy.

Whole exome sequencing: Whole exome or whole genome sequencing (WES/WGS) will be used to identify tumor mutations and copy number variations (CNV), mutational load, neoantigen identification, tumor clonality, TCR clonality, and HLA heterozygosity. Neoantigen identification will allow for us to develop tetramers in the tetramer core which can be applied to patient samples (blood, biopsy and tumor) to identify and phenotype tumor-reactive T cells.

Single cell RNA-seq (scRNA-seq) and analysis algorithms: scRNA-seq has been used and a unique clustering algorithm has been developed to redefine in an unbiased manner and with the highest granularity possible the cellular identity of NSCLC lesions⁴. scRNA-seq will be used to phenotype the immune infiltrate and stroma at the single cell level, as well as analyze the tumor for heterogeneity. Additionally, a novel iteration of this single cell transcriptomic analysis called CITEseq will be used, which incorporates single cell proteomic and transcriptomic analysis using the same 10× platform.

ctDNA: ctDNA will be analyzed to assess correlation with response and/or recurrence. As part of the tumor atlas, patients will have WES performed on their tumors which will allow for detection of tumor-specific DNA in plasma that will be collected in specialized tubes to preserve ctDNA, and cryopreserved over the course of treatment. Given the rapid growth in this field in the past 2 years, analysis will begin once at least 50% of patients have been accrued into the trial, at which point it will be determined which platform will be used to measure ctDNA in plasma taken from patients at time points specified.

### Statistical Considerations

### Analysis of Primary Endpoints

For the six patient Phase 1b run-in portion of the study, the primary endpoint is the occurrence of DLTs as defined in section 9.3, based on the NCI Common Toxicity Criteria for Adverse Events (CTCAE), version 5.0. The design is a modified 3+3 design.

Radiographic response rate will be determined by the RECIST v1.1²⁶. The primary endpoint of the Phase 2 trial is ORR at first imaging following initiation of therapy. The response rate, defined as the percent of patients achieving a CR or PR on initial imaging, will be calculated and a two-sided 80% confidence interval (CI) will be constructed using the methods and software introduced by Koyama and Chen which accounts for the group sequential nature of the design⁴³. The study population consists of all subjects who enroll (including the Phase 1b run-in portion) and receive at least one dose of dupilumab. Following the non-responder imputation method, subjects who do not have an imaging evaluation will be assumed to be non-responders in the analysis of the primary endpoint ORR.

### Analysis of Secondary Endpoints

The analysis cohort for secondary endpoints consists of all subjects who enroll (including the Phase 1b run-in portion) and receive at least one dose of Dupilumab.

The best overall response rate, defined as the percent of patients achieving a CR or PR on initial or subsequent imaging before initiation of subsequent line of anti-cancer therapy, will be calculated and a two-sided 80% confidence interval (CI) will be constructed by the method discussed in section 14.1⁴³. Following the non-responder imputation method, subjects who do not have an imaging evaluation will be assumed to be non-responders in the analysis of the BORR.

Toxicity will be assessed according to the NCI Common Toxicity Criteria for Adverse Events (CTCAE), version 5.0. These data will be summarized using descriptive statistics. All AEs will be summarized with frequency and severity tabulated. Stopping rules are based on DLTs, but all adverse events including clinical laboratory results, and vital signs will be summarized descriptively. All reported AEs will be grouped by organ system class and grade. Clinical laboratory results will be summarized by calculating means, medians, ranges, and standard deviations.

Overall survival (OS) is defined as the time from the first dosing of dupilumab and date of death. Progression-free survival (PFS) is defined as the time from first dosing of dupilumab to date of objectively documented progression of disease or death. Duration of Response (DOR) is defined as the time between the date of first response of CR or PR to the date of first documented tumor progression or death due to any cause. DOR is defined only for patients demonstrating a response. OS, PFS, and DOR will be analyzed using the Kaplan-Meier method.

Statistical analyses for secondary endpoints will conducted by the use of the SAS statistical software.

### Analysis of Exploratory Endpoints

Single cell and tissue-level immune analysis, and creation of dynamic atlases are highly descriptive and largely exploratory in nature; given the novelty of some of the assays that will be incorporated—that are being pioneered by ISMMS investigators and our collaborators—the ability to predict the data that will be revealed is limited. Paired tissue analyses within patients (pre-treatment biopsy vs. on treatment biopsy) will be conducted on the frequency of the tumor immune microenvironment subsets (NK cells, Treg, CD141+DC, clonal PD1+ T cells, etc.) by performing paired t-tests or Wilcoxon signed rank tests if data are not normally distributed. From our similar project in NSCLC⁴, an important finding was that CD8+Granzyme B+ T cells was significantly decreased in frequency in the tumor site (mean=1.5%) compared to normal lung tissue from the same patients (mean=4%) (sd of differences=3%). Several other immune microenvironment subsets had similar differences and effect sizes. For our present study, we anticipate no issue accruing 21 patients for analysis. A sample of 21 patients would allow detection of a mean difference of this magnitude, assuming a similar standard deviation as in the previous study, with power >80% for a two-sided test at the <0.05 level.

Subjects will participate in an exploratory study evaluating the impact of circulating tumor DNA (ctDNA) at separate time points before treatment, and during treatment at the times specified. A repeated measures mixed effects model will be used to estimate the change in ctDNA over time.

Statistical analyses for the exploratory endpoints will be conducted by the SAS statistical software.

### Sample Size and Accrual

This is largely a signal-finding exercise, in which it will be determined whether there is any pathologic effect of the addition of dupilumab to PD-(L)1 blockade. It is postulated that a response rate of 20% or more would be very interesting (H1), and potentially practice changing in the treatment of relapsed/refractory NSCLC for which there are few good treatment options, while an efficacy rate of 5% or less would not suggest a significant therapeutic potential (Ho)

Using this design, initiation of Phase II is probable if the risk of DLT is low, and the likelihood of initiation decreases as the risk of DLT increases, as demonstrated in the Table below.

| | | | | | | |
|---|---|---|---|---|---|---|
| True Risk of Toxicity | 0.01 | 0.20 | 0.30 | 0.40 | 0.50 | 0.60 |
| Probability of Phase | 0.88 | 0.66 | 0.42 | 0.23 | 0.11 | 0.04 |
| II Initiation | | | | | | |

The following table shows the width of 95% confidence intervals calculated for various possible observed DLT rates from the trial.

| Number of Patients with | | 95% Confidence |
|---|---|---|
| DLTs/Number of Patients | DLT Rate | Interval* |
| 0/6 | 0 | [0, 0.39] |
| 1/6 | 0.17 | [0.03, 0.56] |
| 2/6 | 0.33 | [0.10, 0.70] |
| 2/3 | 0.67 | [0.21, 0.94] |

*Wilson Method for computing confidence interval

Given that any responses to the anti-PD-(L)1 backbone of this therapy would not be expected, given patients have to have progressed on prior anti-PD-(L)1 therapy, the operating characteristics for 21 patients will test a null hypothesis of poor overall response of 5% or less versus an alternative hypothesis of a promising response rate of 20% or more at a 10% one-sided significance level and 80% power. A minimax two-stage design will be used. In the first stage, 12 patients will be entered. If no patient has a response (CR or PR), then the study will be terminated. If there is at least one responding patient in the first 12 patients, then 9 additional patients will be accrued. If the total number of patients that have a response is 3 or more, the null hypothesis is rejected and the treatment is recommended for further study. This design has a probability of early termination of 0.54 and was obtained by use of the PASS 2019 v 19.0.2 statistical software.

In addition to the embodiments expressly described herein, it is to be understood that all of the features disclosed in this disclosure may be combined in any combination (e.g., permutation, combination). Each element disclosed in the disclosure may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, and can make various changes and modifications of the invention to adapt it to various usages and conditions. Thus, other embodiments are also within the claims.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or embodiments of the disclosure, is/are referred to as comprising particular elements and/or features, certain embodiments of the disclosure or embodiments of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the present disclosure, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

### REFERENCES

1. Diakos C I, Charles K A, McMillan D C, Clarke S J. Cancer-related inflammation and treatment effectiveness. Lancet Oncol. 2014; 15(11): e493-503.
2. Mantovani A, Allavena P, Sica A, Balkwill F. Cancer-related inflammation. Nature. 2008; 454(7203):436-444.
3. Binnewies M, Roberts E W, Kersten K, et al. Understanding the tumor immune microenvironment (TIME) for effective therapy. Nat Med. 2018; 24(5):541-550.
4. Lavin Y, Kobayashi S, Leader A, et al. Innate Immune Landscape in Early Lung Adenocarcinoma by Paired Single-Cell Analyses. Cell. 2017; 169(4):750-765 e717.
5. Broz M L, Binnewies M, Boldajipour B, et al. Dissecting the Tumor Myeloid Compartment Reveals Rare Activating Antigen-Presenting Cells Critical for T Cell Immunity. Cancer Cell. 2014; 26(6):938.
6. Salmon H, Idoyaga J, Rahman A, et al. Expansion and Activation of CD103 (+) Dendritic Cell Progenitors at the Tumor Site Enhances Tumor Responses to Therapeutic PD-L1 and BRAF Inhibition. Immunity. 2016; 44(4):924-938.
7. Garris C S, Arlauckas S P, Kohler R H, et al. Successful Anti-PD-1 Cancer Immunotherapy Requires T Cell-Dendritic Cell Crosstalk Involving the Cytokines IFN-gamma and IL-12. Immunity. 2018; 49(6):1148-1161 e1147.
8. Coussens L M, Zitvogel L, Palucka A K. Neutralizing tumor-promoting chronic inflammation: a magic bullet? Science. 2013; 339(6117):286-291.
9. Mantovani A, Allavena P. The interaction of anticancer therapies with tumor-associated macrophages. J Exp Med. 2015; 212(4):435-445.
10. Strid J, Sobolev O, Zafirova B, Polic B, Hayday A. The intraepithelial T cell response to NKG2D-ligands links lymphoid stress surveillance to atopy. Science. 2011; 334(6060):1293-1297.
11. Dalessandri T, Strid J. Beneficial autoimmunity at body surfaces-immune surveillance and rapid type 2 immunity regulate tissue homeostasis and cancer. Front Immunol. 2014; 5:347.
12. Maier B, Leader A M, Chen S T, et al. A conserved dendritic-cell regulatory program limits antitumour immunity. Nature. 2020; 580(7802):257-262.
13. Xue W, Meylan E, Oliver T G, et al. Response and resistance to NF-kappaB inhibitors in mouse models of lung adenocarcinoma. Cancer Discov. 2011; 1(3):236-247.
14. Bachert C, Mannent L, Naclerio R M, et al. Effect of Subcutaneous Dupilumab on Nasal Polyp Burden in Patients With Chronic Sinusitis and Nasal Polyposis: A Randomized Clinical Trial. JAMA. 2016; 315(5):469-479.
15. Beck L A, Thaci D, Hamilton J D, et al. Dupilumab treatment in adults with moderate-to-severe atopic dermatitis. The New England journal of medicine. 2014; 371(2):130-139.
16. Castro M, Corren J, Pavord I D, et al. Dupilumab Efficacy and Safety in Moderate-to-Severe Uncontrolled Asthma. The New England journal of medicine. 2018; 378(26):2486-2496.
17. Rabe K F, Nair P, Brusselle G, et al. Efficacy and Safety of Dupilumab in Glucocorticoid-Dependent Severe Asthma. The New England journal of medicine. 2018; 378(26):2475-2485.
18. Simpson E L, Bieber T, Guttman-Yassky E, et al. Two Phase 3 Trials of Dupilumab versus Placebo in Atopic Dermatitis. The New England journal of medicine. 2016; 375(24):2335-2348.
19. Wenzel S, Ford L, Pearlman D, et al. Dupilumab in persistent asthma with elevated eosinophil levels. The New England journal of medicine. 2013; 368(26):2455-2466.
20. Antonia S J, Villegas A, Daniel D, et al. Durvalumab after Chemoradiotherapy in 20. Stage III Non-Small-Cell Lung Cancer. The New England journal of medicine. 2017.
21. Gadgeel S, Rodríguez-Abreu D, Speranza G, et al. Updated analysis from 21. KEYNOTE-189: pembrolizumab or placebo plus pemetrexed and platinum for previously untreated metastatic nonsquamous non-small-cell lung cancer. 2020.
22. Hellmann M D, Paz-Ares L, Bernabe Caro R, et al. Nivolumab plus ipilimumab in advanced non-small-cell lung cancer. New England Journal of Medicine. 2019; 381(21):2020-2031.
23. Paz-Ares L, Luft A, Vicente D, et al. Pembrolizumab plus chemotherapy for squamous non-small-cell lung cancer. New England Journal of Medicine. 2018; 379(21):2040-2051.
24. Duan J, Cui L, Zhao X, et al. Use of immunotherapy with programmed cell death 1 vs programmed cell death ligand 1 inhibitors in patients with cancer: a systematic review and meta-analysis. JAMA oncology. 2020; 6(3):375-384.
25. Wang Y, Zhou S, Yang F, et al. Treatment-related adverse events of PD-1 and PD-L1 inhibitors in clinical trials: a systematic review and meta-analysis. JAMA oncology. 2019; 5(7):1008-1019.
26. Eisenhauer E A, Therasse P, Bogaerts J, et al. New response evaluation criteria in solid tumours: revised RECIST guideline (version 1.1). Eur J Cancer. 2009; 45(2):228-247.
27. Bachert C, Han J K, Desrosiers M, et al. Efficacy and safety of dupilumab in patients with severe chronic rhinosinusitis with nasal polyps (LIBERTY NP SINUS-24 and LIBERTY NP SINUS-52): results from two multicentre, randomised, double-blind, placebo-controlled, parallel-group phase 3 trials. The Lancet. 2019; 394(10209):1638-1650.
28. Rabe K F, Nair P, Brusselle G, et al. Efficacy and safety of dupilumab in glucocorticoid-dependent severe asthma. New England Journal of Medicine. 2018; 378(26):2475-2485.
29. Brahmer J R, Lacchetti C, Schneider B J, et al. Management of immune-related adverse events in patients treated with immune checkpoint inhibitor therapy: American Society of Clinical Oncology Clinical Practice Guideline. Journal of clinical oncology: official journal of the American Society of Clinical Oncology. 2018; 36(17):1714.
30. Puzanov I, Diab A, Abdallah K, et al. Managing toxicities associated with immune checkpoint inhibitors: consensus recommendations from the Society for Immunotherapy of Cancer (SITC) Toxicity Management Working Group. Journal for immunotherapy of cancer. 2017; 5(1):1-28.
31. Ivanova A, Qaqish B F, Schell M J. Continuous toxicity monitoring in phase II trials in oncology. Biometrics. 2005; 61(2):540-545.
32. Angelo M, Bendall S C, Finck R, et al. Multiplexed ion beam imaging of human breast tumors. Nat Med. 2014; 20(4):436-442.
33. Chowell D, Morris L G T, Grigg C M, et al. Patient HLA class I genotype influences cancer response to checkpoint blockade immunotherapy. Science. 2017: In press.
34. Danilova L, Wang H, Sunshine J, et al. Association of PD-1/PD-L axis expression with cytolytic activity, mutational load, and prognosis in melanoma and other solid tumors. Proceedings of the National Academy of Sciences of the United States of America. 2016; 113(48): E7769-E7777.
35. Ho A S, Kannan K, Roy D M, et al. The mutational landscape of adenoid cystic carcinoma. Nature Genetics. 2013; 45(7):791-798.
36. Łuksza M, Riaz N, Makarov V, et al. A neoantigen fitness model predicts tumour response to checkpoint blockade immunotherapy. Nature. 2017; 551(7681):517-520.
37. Remark R, Merghoub T, Grabe N, et al. In-depth tissue profiling using multiplexed immunohistochemical consecutive staining on single slide. Science Immunology. 2016; 1(1): aaf6925.
38. Riaz N, Havel J J, Makarov V, et al. Tumor and Microenvironment Evolution during Immunotherapy with Nivolumab. Cell. 2017; 171(4):934-949 e915.
39. Rizvi N A, Hellmann M D, Snyder A, et al. Cancer immunology. Mutational landscape determines sensitivity to PD-1 blockade in non-small cell lung cancer. Science. 2015; 348(6230):124-128.
40. Snyder A, Makarov V, Merghoub T, et al. Genetic basis for clinical response to CTLA-4 blockade in melanoma. N Engl J Med. 2014; 371(23):2189-2199.
41. Yarchoan M, Xing D, Luan L, et al. Characterization of the immune microenvironment in hepatocellular carcinoma. Clin Cancer Res. 2017; 23(23):7333-7339.
42. Hectors S J, Wagner M, Bane O, et al. Quantification of hepatocellular carcinoma heterogeneity with multiparametric magnetic resonance imaging. Sci Rep. 2017; 7(1):2452.
43. Koyama T and Chen H. Proper inference from Simon's two-stage designs. Stat Med. 2008; 27(16):3145-3154.

What is claimed is:

1. A method of treating cancer comprising administering to a subject a first dose of about 600 mg of dupilumab followed by one or more successive doses of about 300 mg of dupilumab about every three weeks thereafter, wherein the subject was treated or is undergoing treatment with a PD-1 or PD-L1 blocking agent.

2. The method of claim 1, wherein the cancer is a solid tumor.

3. The method of any one of the above claims, wherein the cancer is non-small cell lung cancer (NSCLC).

4. The method of any one of the above claims, wherein the cancer is relapsed/refractory metastatic NSCLC.

5. The method of any one of the above claims, wherein the treatment with the PD-1 or PD-L1 blocking agent is a standard of care treatment.

6. The method of any one of the above claims, wherein the PD-1 or PD-L1 blocking agent is selected from the group consisting of nivolumab, pembrolizumab, pidilizumab, MEDI0680, durvalumab, and combinations thereof.

7. The method of any of the above claims, wherein the PD-1 or PD-L1 blocking agent is durvalumab.

8. The method of any of the above claims, wherein the PD-1 or PD-L1 blocking agent is pembrolizumab.

9. The method of any of the above claims, wherein the PD-1 or PD-L1 blocking agent is nivolumab.

10. The method of any one of the above claims, wherein the subject is further administered an anti-CTLA-4 immune checkpoint inhibitor therapy selected from the group consisting of ipilimumab, tremelimumab, and combinations thereof.

11. The method of claim 9, further comprising administering a therapeutically effective amount of ipilimumab.

12. The method of any of the above claims, wherein the first dose of dupilumab is administered on day 1.

13. The method of any one of the above claims, wherein the one or more successive doses of about 300 mg of dupilumab is one successive dose of about 300 mg of dupilumab.

14. The method of claim 13, wherein the one successive dose of dupilumab is administered on day 22 of the method.

15. The method of any one of the above claims, wherein the one or more successive doses of about 300 mg of dupilumab is two successive doses of about 300 mg of dupilumab.

16. The method of claim 15, wherein the two successive doses of dupilumab are administered on day 22 and day 43, respectively.

17. The method of claim 1, wherein the subject is administered a first dose of 600 mg of dupilumab on day 1, a second dose of 300 mg of dupilumab on day 22, and a third dose of 300 mg of dupilumab on day 43.

18. The method of any one of the above claims, wherein the administering of the first dose of dupilumab and/or the one or more successive doses are by intravenous administration.

19. The method of any one of the above claims, wherein the cancer is selected from the group consisting of: bladder cancer, breast cancer, colon and rectal cancer, endometrial cancer, kidney or renal cell cancer, leukemia, lung cancer, melanoma, Non-Hodgkin lymphoma, pancreatic cancer, prostate cancer, ovarian cancer, stomach cancer, wasting disease, and thyroid cancer.

20. The method of any one of the above claims, wherein the cancer is a cardiac cancer selected from the group consisting of: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hanlartoma, inesothelioma.

21. The method of any one of the above claims, wherein the cancer is a gastrointestinal cancer selected from the group consisting of: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma).

22. The method of any one of the above claims, wherein the cancer is a genitourinary cancer selected from the group consisting of: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma).

23. The method of any one of the above claims, wherein the cancer is a liver cancer selected from the group consisting of: hepatoma (hepatocellular carcinoma), cholangiocarcinoma, hepatoblastoma, angiosarcoma, hepatocellular adenoma, hemangioma.

24. The method of any one of the above claims, wherein the cancer is a bone cancer selected from the group consisting of: osteogenic sarcoma (osteosarcoma), fibrosarcoma, malignant fibrous histiocytoma, chondrosarcoma, Ewing's sarcoma, soft tissue Ewing's sarcoma, soft tissue sarcoma, synovial sarcoma, malignant lymphoma (reticulum cell sarcoma), multiple myeloma, malignant giant cell tumor chordoma, desmoid-type fibromatosis, fibroblastic sarcoma, gastrointestinal stromal tumors, retroperitoneal sarcoma, osteochronfroma (osteocartilaginous exostoses), benign chondroma, chondroblastoma, chondromyxofibroma, osteoid osteoma and giant cell tumors.

25. The method of any one of the above claims, wherein the cancer is a nervous system cancer selected from the group consisting of: osteoma, hemangioma, granuloma, xanthoma, osteitis defomians, meningioma, meningiosarcoma, gliomatosis, astrocytoma, medulloblastoma, glioma, ependymoma, germinoma, glioblastoma multiforme, oligodendroglioma, schwannoma, retinoblastoma, congenital tumors, spinal cord neurofibroma, meningioma, glioma, sarcoma.

26. The method of any one of the above claims, wherein the cancer is a hematologic cancer selected from the group consisting of: myeloid leukemia, acute lymphoblastic leukemia, chronic lymphocytic leukemia, myeloproliferative diseases, multiple myeloma, myelodysplastic syndrome, Hodgkin's disease, non-Hodgkin's lymphoma.

27. The method of any one of the above claims, wherein the cancer is a skin cancer selected from the group consisting of: malignant melanoma, basal cell carcinoma, squamous cell carcinoma, Karposi's sarcoma, moles, dysplastic nevi, lipoma, angioma, dermatofibroma, keloids, psoriasis.

28. A method of augmenting an immune checkpoint therapy in a subject having cancer, comprising administering to the subject a first dose of about 600 mg of dupilumab followed by one or more successive doses of about 300 mg of dupilumab about every three weeks thereafter, wherein the immune checkpoint therapy comprises a PD-(L)1 blocking agent.

29. The method of claim 28, wherein the cancer is a solid tumor.

30. The method of claim 28 or 29, wherein the cancer is non-small cell lung cancer (NSCLC).

31. The method of any one of claims 28-30, wherein the cancer is relapsed/refractory metastatic NSCLC.

32. The method of any one of claims 28-31, wherein the treatment with the PD-1 or PD-L1 blocking agent is a standard of care treatment.

33. The method of any one of claims 28-32, wherein the PD-(L)1 blocking agent is selected from the group consisting of nivolumab, pembrolizumab, pidilizumab, MEDI0680, durvalumab, and combinations thereof.

34. The method of any one of claims 28-33, wherein the PD-(L)1 blocking agent is durvalumab.

35. The method of any one of claims 28-33, wherein the PD-(L)1 blocking agent is pembrolizumab.

36. The method of any one of claims 28-33, wherein the PD-(L)1 blocking agent is nivolumab.

37. The method of any one of claims 28-36, wherein the subject is further administered an anti-CTLA-4 immune checkpoint inhibitor therapy selected from the group consisting of ipilimumab, tremelimumab, and combinations thereof.

38. The method of claim 37, further comprising administering a therapeutically effective amount of ipilimumab.

39. The method of any one of claims 28-38, wherein the first dose of dupilumab is administered on day 1.

40. The method of any one of claims 28-39, wherein the one or more successive doses of about 300 mg of dupilumab is one successive dose of about 300 mg of dupilumab.

41. The method of claim 40, wherein the one successive dose of dupilumab is administered on day 22 of the method.

42. The method of any one of claims 28-39, wherein the one or more successive doses of about 300 mg of dupilumab is two successive doses of about 300 mg of dupilumab.

43. The method of claim 42, wherein the two successive doses of dupilumab are administered on day 22 and day 43, respectively.

44. The method of claim 28, wherein the subject is administered a first dose of 600 mg of dupilumab on day 1, a second dose of 300 mg of dupilumab on day 22, and a third dose of 300 mg of dupilumab on day 43.

45. The method of any one of claims 28-44, wherein the administering of the first dose of dupilumab and/or the one or more successive doses are by intravenous administration.

46. The method of claim 28, wherein the cancer is selected from the group consisting of: bladder cancer, breast cancer, colon and rectal cancer, endometrial cancer, kidney or renal cell cancer, leukemia, lung cancer, melanoma, Non-Hodgkin lymphoma, pancreatic cancer, prostate cancer, ovarian cancer, stomach cancer, wasting disease, and thyroid cancer.

47. The method of claim 28, wherein the cancer is a cardiac cancer selected from the group consisting of: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hanlartoma, inesothelioma.

48. The method of claim 28, wherein the cancer is a gastrointestinal cancer selected from the group consisting of: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma).

49. The method of claim 28, wherein the cancer is a genitourinary cancer selected from the group consisting of: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma).

50. The method of claim 28, wherein the cancer is a liver cancer selected from the group consisting of: hepatoma (hepatocellular carcinoma), cholangiocarcinoma, hepatoblastoma, angiosarcoma, hepatocellular adenoma, hemangioma.

51. The method of claim 28, wherein the cancer is a bone cancer selected from the group consisting of: osteogenic sarcoma (osteosarcoma), fibrosarcoma, malignant fibrous histiocytoma, chondrosarcoma, Ewing's sarcoma, soft tissue Ewing's sarcoma, soft tissue sarcoma, synovial sarcoma, malignant lymphoma (reticulum cell sarcoma), multiple myeloma, malignant giant cell tumor chordoma, desmoid-type fibromatosis, fibroblastic sarcoma, gastrointestinal stromal tumors, retroperitoneal sarcoma, osteochronfroma (osteocartilaginous exostoses), benign chondroma, chondroblastoma, chondromyxofibroma, osteoid osteoma and giant cell tumors.

52. The method of claim 28, wherein the cancer is a nervous system cancer selected from the group consisting of: osteoma, hemangioma, granuloma, xanthoma, osteitis defomians, meningioma, meningiosarcoma, gliomatosis, astrocytoma, medulloblastoma, glioma, ependymoma, germinoma, glioblastoma multiforme, oligodendroglioma, schwannoma, retinoblastoma, congenital tumors, spinal cord neurofibroma, meningioma, glioma, sarcoma.

53. The method of claim 28, wherein the cancer is a hematologic cancer selected from the group consisting of: myeloid leukemia, acute lymphoblastic leukemia, chronic lymphocytic leukemia, myeloproliferative diseases, multiple myeloma, myelodysplastic syndrome, Hodgkin's disease, non-Hodgkin's lymphoma.

54. The method of claim 28, wherein the cancer is a skin cancer selected from the group consisting of: malignant melanoma, basal cell carcinoma, squamous cell carcinoma, Karposi's sarcoma, moles, dysplastic nevi, lipoma, angioma, dermatofibroma, keloids, psoriasis.

55. A pharmaceutical kit for treating cancer in a subject comprising a first dose of dupilumab of 600 mg, a second dose of dupilumab of 300 mg, and a third dose of dupilumab of 300 mg, and a pharmaceutically acceptable excipient, optionally one or more doses of a PD-1(L) blocking agent, and a pharmaceutically acceptable excipient.

56. The pharmaceutical kit of claim 55, further comprising one or more medical devices capable of delivering the first, second, and third doses of dupilumab, and optionally the PD-1(L) blocking agent intravenously.
